(19) United States
(12) Patent Application Publication
(10) Pub. No.: US 2009/0186929 A1
(43) Pub. Date: Jul. 23, 2009

(54) Method and agent for inducing apoptosis/cell death in leukemia cells

(75) Inventors: Joe Brice Weinberg, Durham, NC (US); Marc Christopher Levesque, Sewickley, PA (US); Dipak Kumar Ghosh, Durham, NC (US)

Correspondence Address: DINESH AGARWAL, P.C., 5350 SHAWNEE ROAD, SUITE 330, ALEXANDRIA, VA, 22312, US

(73) Assignee: U.S. DEPARTMENT OF VETERANS AFFAIRS, WASHINGTON, DC (US)

(21) Appl. No.: 12/318,349

(22) Filed: Dec. 29, 2008

Related U.S. Application Data
(63) Continuation-in-part of application No. 10/983,978, filed on Nov. 9, 2004.
(60) Provisional application No. 60/518,304, filed on Nov. 10, 2003.

(51) Int. Cl. A61K 31/4164; A61K 31/416; A61K 31/381; A61K 31/155; A61P 35/02

(52) U.S. Cl. 514/396; 514/403; 514/448; 514/637

(57) ABSTRACT

A method for inducing apoptosis or cell death in leukemia cells includes inhibiting the production of nitric oxide (NO) by using a nitric oxide synthase (NOS) inhibitor. The NOS inhibitor includes a NOS1-specific inhibitor, such as N-[4-(2-{[(3-chlorophenyl)methyl]amino}ethyl)phenyl]-2-thiophenecarboximide dihydrochloride, [N⁵-(1-imino-3-butenyl)-L-ornithine], 7-nitroindazole, 1-(2-trifluoromethylphenyl)imidazole, 3-bromo-7-nitroindazole, and S-ethyl-N-[4-(trifluoromethyl)phenyl)isothiourea HCl.

### CROSS-REFERENCE TO RELATED APPLICATIONS

This is a continuation-in-part of application Ser. No. 10/983,978, filed Nov. 9, 2004, which claims the benefit of prior U.S. Provisional Application Ser. No. 60/518,304, filed Nov. 10, 2003, both of which are hereby incorporated herein in their entirety by reference.

### STATEMENT REGARDING FEDERALLY SPONSORED RESEARCH OR DEVELOPMENT

The work leading to the present invention was supported by one or more grants from the U.S. Government, including NIH grant No. NIH-RO-1CA 90548 and the Department of Veterans Affairs Merit Review Grants. The U.S. Government therefore has certain rights in the invention.

### FIELD AND BACKGROUND OF THE INVENTION

The present invention is generally directed to the treatment of cancer, and more particularly to a method and agent for inducing apoptosis/cell death in leukemia cells.

Chronic lymphocytic leukemia (CLL) is the most common form of leukemia in North America and Europe, accounting for more than 30% of all cases. CLL is characterized by the accumulation of non-dividing CD5⁺ B lymphocytes in G₀ of the cell cycle. Although treatments exist for this disease, it is essentially an incurable malignancy. There is a great need for new insight into disease mechanisms, and development of new treatments.

There has been much progress in NO (nitric oxide) biology research since the late 1980s when NO was discovered as a mediator of macrophage-mediated tumor cytotoxicity, vessel dilation, and neurotransmission. We know that NO has effects in essentially all fields of biology. Likewise, there also has been much progress in CLL research over the past 10 years, with more understanding of the control of CLL cell life/death and more effective therapy.

CLL typically occurs in older patients (highest in those aged 55 to 70 years), with only 20% younger than 55 years (References 2 and 3). There is evidence of genetic susceptibility, with reports of familial CLL not uncommon. CLL affects men twice more often than women (Reference 3). Autoimmunity is common (being manifest primarily as presence of polyclonal antibody formation against cell membrane antigens), despite the near universal finding of hypogammaglobulinemia. Staging systems of CLL incorporate physical findings (lymphadenopathy and hepatosplenomegaly) and hematological parameters (hemoglobin levels and platelet count). Patients with early stage CLL may not benefit from therapy, but as they progress to have anemia, thrombocytopenia, and systemic symptoms and signs, treatment is helpful. The median survival with our best treatments is about 10 years, with deaths coming directly from CLL complications or from secondary malignancies (Reference 3). Treatments for CLL, though generally non-curative, are effective at palliating symptoms and avoiding complications of disease, and they likely prolong life (Reference 3). Mainstays of treatment have been alkylating agents (especially chlorambucil)±glucocorticoids, with no documented benefit of anthracyclines. The nucleoside analogue fludarabine is an especially important drug for CLL. Other nucleoside analogues (e.g., deoxycoformycin and 2-chlorodeoxyadenosine) are also active in CLL, but less so than fludarabine. Anti-CD20 (rituximab) and anti-CD52 antibody (alemtuzumab) therapy are effective as salvage therapy (either alone or in combination). Anti-CD52 antibody appears to be unique with a high proportion of complete responders being noted (Reference 4). Bone marrow transplant for CLL is still considered experimental. Despite encouraging results with nucleoside analogues, essentially all CLL patients die with disease. New treatments are needed.

CLL is characterized by accumulation of non-dividing CD5+ B cells in G₀ of the cell cycle. Although CLL cells are long-lived in vivo, they undergo rapid and spontaneous apoptosis when cultured in vitro suggesting that viability of CLL cells is dependent on a factor(s) that is absent ex vivo. NO is an important regulator of apoptosis (References 5-8). New information suggests that viability of cultured CLL cells may be dependent on the autocrine, endogenous production of NO (References 9-11).

The leukemic cells express mature B lymphocyte antigens (e.g., CD19 and CD20), and characteristically CD5. Surface immunoglobulins (and the B cell antigens) are present at low levels, probably due to defects in CD79b caused by alternative splicing of mRNA. The origin and fate of CD5+ B cells in humans is not fully understood. CD5+ B cells are present in increased numbers in normal human cord blood, fetal spleen and in the blood of patients after bone marrow transplant. In normal adults, they are also found in low numbers in the blood, the tonsils, and the mantle zone of secondary follicles in lymph nodes. Normal CD5+ B cells can develop into functionally active macrophage-like cells with expression of myeloid markers and a cytoskeletal organization similar to macrophages (Reference 12). CD5+ B cells have been associated with the production of polyreactive IgM autoantibodies that use a restricted repertoire of non-mutated Ig V genes.

Bcl-2 is an anti-apoptotic protein; its levels decrease with in vitro culture of CLL cells; bcl-2/bax ratios correlate inversely with susceptibility of cultured CLL cells to undergo spontaneous and drug-induced apoptosis. Bcl-2 levels in CLL cells are inversely correlated with CLL patient survival.

Several genetic irregularities have been noted in CLL, with abnormalities occurring in more than 80% of patients (Reference 13). Some of these molecular features correlate strongly with CLL severity. Deletions or translocations at 13q are the most common. This abnormality is associated with a relatively benign course. Patients with trisomy 12, the second most common abnormality, have aggressive, rapidly progressive disease. Deletion in chromosome bands 11q22-q23 [most likely the ataxia telangiectasia mutated (ATM) gene] is the third most common chromosome aberration in CLL. The frequent somatic disruption of both alleles of the ATM gene in CLL by deletion or point mutation indicates a possible pathogenic role in CLL. The mutations appear to be somatic in origin. ATM mutation is associated with extensive lymph node involvement and poor survival. Mutations of p53 at 17p13.3 are seen in 15-30% of patients with CLL. They may be associated with a more aggressive form disease and propensity to develop Richter's syndrome (transformation into, or acquisition of, aggressive non-Hodgkin's lymphoma). Short telomere length and high telomerase activity are significantly associated with shorter survival in CLL.

Immunoglobulin heavy chain mutation status and CD38 expression correlate closely with prognosis in CLL (References 14 and 15). Irrespective of stage of disease, those with unmutated V-H immunoglobulin chains and high CD38 expression (defined as >30% of cells positive) have a shortened survival. For example, in early stage disease, patients with unmutated V-H chains have a median survival of 95 months, while in those with mutated V-H chains, this is 293 months (Reference 14).

Microarray studies have revealed that CLL cells are likely derived from “memory B cells” or “activated B cells,” and that they display unique patterns of gene expression (References 16 and 17). Jelinek and co-workers, using microarray analysis, demonstrated a group of 31 genes that distinguished between low and high risk patients, suggesting that there may be a unique gene expression signature that associates with diseases expression (Reference 18). The zeta-chain associated chain of the T cell receptor (Zap70) is overexpressed in CLL cells (microarray, quantitative mRNA, protein by flow cytometry, histochemistry, and immunoblot). Zap70 expression closely correlates with the presence of unmutated immunoglobulin H chains, and with a poor prognosis (References 19 and 20). Microarray and Zap70 analyses, as well as other clinically convenient testing such as lymphocyte doubling time, beta-2 microglobulin, chromosome analyses, and serum thymidine kinase levels serve as important prognostic variables (Reference 21). However, the immunoglobulin heavy and light gene somatic mutation status remains the most powerful and the most difficult to perform test (Reference 21).

Several endogenous factors prevent spontaneous apoptosis of cultured CLL cells. Among the factors are IFN-α, IFN-γ, G-CSF, IL-2, IL-4, IL-6, IL-8, IL-13, CD40 ligation, CD6 ligation, and contact with bone marrow-derived stromal cells (References 22 and 23). IL-4, IL-8 and CD6 ligation may prevent spontaneous apoptosis of cultured CLL cells by maintaining cellular bcl-2 levels. Factors capable of inducing CLL cell proliferation include IL-2 and CD40 ligand. IL-5 and IL-10 promote apoptosis of CLL cells in vitro. Several of the factors (e.g., IL-6, IL-8, IL-10 and IFN-γ are produced by CLL cells. IL-6, IL-10, IFN-γ, and CD40 ligand have been found in serum of patients with CLL (References 22 and 23).

Apoptosis is controlled in part by balances of pro- and anti-apoptotic factors. Bcl-2 belongs to a family of genes that have interrelated roles in apoptosis. Bcl-2 inhibits apoptosis, while bax enhances it. Bcl-xL synergizes with bcl-2, while bcl-xS inhibits bcl-2 function. CLL cells express high levels of bcl-2, bcl-xL, and bax, while bcl-xS is very low in most cases (References 22 and 23). CLL cells do not express the pro-apoptotic molecules Fas (CD95) or c-myc.

A variety of chemotherapy drugs induce apoptosis of neoplastic cells (including CLL cells) (Reference 3). While fludarabine is incorporated into DNA of proliferating cells, it is also toxic for nondividing cells (such as CLL cells). Janus kinases (Jak) and signal transducer and activator of transcription (STAT) factors are important in mediating the cellular activity of various cytokines including interferons. Frank and co-workers noted that CLL cells from 32/32 patients contained STAT1 and STAT3 constitutively phosphorylated on serine residues, whereas B lymphocytes from normals did not (Reference 24). Recent work has demonstrated that fludarabine (but not deoxycoformycin or cyclosporine A) potently and selectively inhibits STAT1 signaling. When resting or activated normal blood lymphocytes are treated in vitro with fludarabine, there is a dramatic and persistent decrease of STAT1 activation by IFN-α, IFN-γ, IL-2, and IL-6, and of STAT1-dependent gene transcription. This is associated with specific depletion of STAT1 protein and mRNA. Importantly, this STAT1 loss was noted in lymphocytes taken from a CLL patient who had received in vivo fludarabine 24 hours previously (Reference 25). STAT1 is especially important in the mediation of cytokine-stimulated expression of NOS2. Thus, given that (i) STAT1 is critical for NOS2 expression, (ii) fludarabine specifically diminishes STAT1, and (iii) inhibition of NO production causes death of CLL cells, we have hypothesized that fludarabine might decrease NOS2 expression, and that NOS inhibitors will act cooperatively as potent killers of CLL cells.

There are no good human cell lines that represent CLL. Most claimed do not have the typical CLL phenotype (positive for CD19, CD20, CD5, CD23, with dim surface Ig), and most are positive for EB (Epstein-Barr) virus. Human leukemia cell xenografts grow poorly in immunodeficient mice. While xenogeneic human/mouse models using CLL cells show promise, these cells are very difficult to grow in normal or immunodeficient mice. CLL cells can survive and possibly disseminate in severe combined immunodeficiency disease (SCID) mice (Reference 26). SCID mice lack functional T and B cells, but do have NK cell function. CLL cells in these xenogeneic mice display characteristics of the cells that were noted in the patients (e.g., Ig expression and production, and response to chemotherapy agents). Likewise, certain leukemia cells can be grown well in immunodeficient nonobese diabetic (NOD)/SCID mice. NOD/SCID mice lack B and T cells, and also have no functional NK cells, no circulating complement, and have defects in antigen presenting cells. While NOD mice develop diabetes mellitus, NOD/SCID mice do not. There are reports of successful growth/survival of CLL in SCID mice, but to this point, there are no reports of growth of CLL in NOD/SCID mice. It would be very useful to have a good animal model for study of human CLL, but I consider this model as still developmental, and not fully suitable for the study of NOS inhibitors.

Bichi, et al reported in 2002 that transgenic mice expressing the TCL1 gene targeted to B lymphocytes (directed by the immunoglobulin V_{H} promoter and the Ig_{H}-u enhancer Eu promoter) develop a disease very similar to CLL (Reference 27). TCL1 is an oncogene normally expressed in immature T lymphocytes. In certain T cell malignancies in humans such as T cell leukemia, there is activation of this oncogene by inversions or translocations that juxtapose it to a T cell receptor locus (Reference 28). Mice made transgenic for TCL1 directed to T cells by the Ick promoter develop T cell leukemia (Reference 29). Mice with TCL1 in B lymphocytes develop very high numbers of B220low, Mac1/CD11b+, CD5+, IgM+ leukemia cells consistent in mice with CD5+ B1 B lymphocytes. The leukemia cells are mono- or oligoclonal. By age 13 to 18 months, the mice become ill and overtly leukemic, with the leukemia (cells that are arrested in the G₀/G₁ phase of the cell cycle) accumulating in the bone marrow, spleen, and other organs (Reference 27). The mice develop WBC up to 180,000/uL (normal in mouse being approximately 3,000/uL), and eventually die of disease. The leukemia is transplantable into other mice, so the model lends itself to efficient use in examining a CLL-like disease in mice.

NO is a lipid soluble, gaseous, free radical produced during enzymatic conversion of L-arginine to L-citrulline. NO is unstable within cells with a half-life measured in seconds. The short NO half-life results from its reaction with oxygen, transition metal ions, and thiols (Reference 30). Reaction of NO with oxygen leads to the production of nitrite and nitrate ions, stable catabolites that are readily measured as surrogate markers of NO production (References 30 and 31).

In the presence of oxygen, NO rapidly (seconds) is converted to nitrogen dioxide and then nitrite and nitrate, substances which are generally not bioactive (Reference 30). NO also reacts with O₂—, and O₂— dismutase (SOD) prolongs NO life by eliminating O₂—. NO binds with high affinity to iron in heme groups of proteins such as hemoglobin (Hb), myoglobin (Mb), and guanylyl cyclase. Hb and Mb are very effective quenchers of NO action. On reacting with O₂—, NO forms peroxynitrite, a very toxic and reactive molecule that may actually be one of the most important final effector toxic molecules when one thinks of NO toxicity in oxygenated systems.

NO quenchers/scavengers inhibit the actions of NO in a variety of systems (Reference 32). Effective quenchers include proteins containing heme (e.g., Hb & Mb), iron-containing complexes [e.g., iron-diethylenetriaminepentaacetic acid or iron ferrioxamine B complexes, or ruthenium complexes (Reference 32)], and cobalt-containing compounds (e.g., hydroxocobalamin (Reference 105). Proteins such as Hb generally stay extracellular, while small molecules (cobalamins and chelator-metal complexes, e.g.) enter cells. NO actions in vivo are blocked by quenchers.

NO is produced from L-arginine by three NOS in humans. NOS1 (“neural” NOS) and NOS3 (“endothelial” NOS) generally produce low levels of NO and are constitutively active. In human cells, inducible NOS (NOS2) produces NO in response to several stimuli including IFN-α, IFN-7, IL-1, TNF-x, IL-6 and LPS (Reference 33). IFN-α, IFN-γ and IL-6 also prevent spontaneous apoptosis of cultured CLL cells. This suggests a possible link between the inhibition of spontaneous apoptosis of cultured CLL cells and NO production.

Both NOS2 and NOS3 have been detected in human B cells (References 5, 7, 9-11 and 34). NOS3 mRNA and protein (RT-PCR and histochemistry) have been noted in tonsil-derived B cells and in the Daudi and Raji B cell lines (Reference 34). NO production by the B cells has not been measured; therefore, the functional significance of B cell NOS3 remains unclear. NOS2 mRNA and protein have been detected in EBV-negative and -positive human B lymphoma cell lines (References 5, 7 and 35). NOS2 in these cell lines is functional as evidenced by its ability to produce NO that inhibits reactivation of latent Epstein-Barr virus infection and blocks Fas-mediated apoptosis. Prior to our work, NOS1 had not been reported in CLL cells, although some noted NOS1 expression in non-Hodgkin's lymphoma and myeloma cells (Reference 36).

The role of NO in apoptosis has not been completely defined (Reference 8). NO is the prototypic molecule with dichotomous actions—the “ying/yang,” “good/bad,” “double-edged sword” effect. For example, work by us and others has shown that NO can either induce death of cells or protect cells from death (Reference 8). Macrophage-produced NO was initially identified as the primary effector that caused stasis and lysis of tumor cells (Reference 37). The effects of NO on apoptosis depend on both the cells being studied and the methods and rates of NO administration. As such, some studies have shown that NO induces apoptosis (References 38-40), while other studies have shown that NO inhibits apoptosis (References 5-7). We have noted that delivery of NO from NO pro-drugs in vitro (uM to mM concentrations) to cultures of acute nonlymphocytic leukemia cells (cell line cells and freshly-isolated cells) causes apoptosis and death (References 1 and 39-41). The degree of toxicity is indirectly related to the rate of NO delivery from the pro-drug (higher kill with lower, chronic release rates) (References 39 and 40). NO toxicity for cells may also be related to the origin of the NO (exogenously supplied and endogenously generated) NO may function differently (Reference 42). Overall, it appears that high level NO from extracellular sources causes apoptosis and cell death by a variety of mechanisms including direct membrane damage, inhibition of ribonucleotide reductase, and inhibition of cellular generation of ATP by mitochondrial electron transport enzymes, aconitase, and GAPDH. However, endogenous or low level NO can also inhibit apoptosis by nitrosylating caspases and perhaps by increasing bcl-2 expression.

Apoptosis can be triggered by a variety of mechanisms via the “mitochondria pathway” (e.g., chemotherapy drugs, x-ray therapy, uv irradiation, and withdrawal of growth factors), and via the “death receptor” [e.g., TNF-α, granzyme B, TRAIL and Fas (CD95) ligand]. Apoptosis is mediated through activation of intracellular cysteine-aspartate proteinases (caspases) that are the human homologues of the C. elegans ced-3 and ced-4 enzymes. Anti-apoptotic proteins include those of the bcl-2 family (Reference 43). NO binds to and inhibits the active site of many of the human caspase family members including caspases 3, 8, and 9 (Reference 44). In CLL, there are a variety of caspases and apoptosis inhibitor proteins that may be important in determining spontaneous and drug-induced apoptosis and response to therapy (References 45 and 46). Also, in resting, normal B lymphocytes, the active site cysteine of caspase 3 is nitrosylated (and inhibited by this nitrosylation), and it undergoes denitrosylation upon fas activation and apoptosis (Reference 35). In addition, NO maintains bcl-2 levels in cultured mouse splenic B cells and prevents their spontaneous apoptosis (Reference 6). The relationship of NOS expression and NO production by CLL cells to their caspase activity and bcl-2 expression has not been examined methodically.

DNA damage from a variety of causes [e.g., physical and chemicals mutagens (including NO)] results in p53 accumulation (Reference 47). p53 can activate transcription of growth regulatory genes resulting in growth arrest and probable DNA repair, and p53 may induce apoptosis. Also, p53 serves to reduce expression of NOS2 mRNA and protein (Reference 47). Mice with genetically disrupted p53 have increased expression of NOS2 and overproduce NO in vivo (Reference 48). Studying 118 human colorectal cancers for NOS2 expression and p53 gene mutations, Ambs et al found G:C to A:T p53 mutations in 62% of cases and noted a significant association between this mutation and NOS2 activity when compared with tumors with other types of mutations (Reference 49). These authors note that NO may act as both an endogenous initiator and promoter of carcinogenesis, and suggest that NOS inhibitors may have antitumor activity. Based on our findings, we think that this could be in part mediated by a release of NO-mediated inhibition of apoptosis. The findings of p53 mutation and accumulation in CLL could be related to overexpression of NOS.

NO from NOS1 has been reported to be an important modulator of nervous tissue cell apoptosis. Andoh and colleagues noted that NOS1 influences bcl-2 and other apoptosis regulators, and accounts for some of the neural cell resistance to apoptosis of preconditioning stress (Reference 50). Others showed increased NOS1 in dorsal root ganglion neurons, as well as an NO inhibition of bax and caspases and apoptosis (Reference 51).

The endothelial isoform (NOS3) is constitutive and tightly regulated by calcium and calmodulin. It plays a major role in regulating vascular tone. Inducible NOS(NOS2) is seen in many cell types, but is prototypically noted in macrophages, hepatotypes, and chondrocytes. It is regulated transcriptionally, and its activity is independent of calcium. NOS2 can produce large (uM amounts) of NO. Neuronal NOS (NOS1) is noted in nervous tissue cells, muscle cells, and testicular cells. It is expressed constitutively, and its activity (like NOS3) is tightly regulated by calcium and calmodulin. It produces very small amounts of NO (low nM amounts), levels capable of acting in signaling, for example, but not high enough for other functions such as for cytotoxicity.

While NOS1 and NOS3 are thought of as constitutive enzymes, both can be also regulated at the level of transcription. Regulation of NOS2 occurs primarily transcriptionally, but the regulation of NOS2 mRNA can occur at multiple steps (Reference 52) including mRNA transcription, mRNA stability, and mRNA translation. Over the last 5 years, we have done detailed investigations of NOS2 promoter polymorphisms regarding their functional significance and relationship to disease in humans. In the first 7.3 kb of the promoter, we identified 34 unique SNPs and inferred 71 SNP (single nucleotide polymorphism) haplotypes. Certain SNPs and haplotypes are significantly associated with increased NO production in vivo in humans, and with protection from severe malaria (References 53 and 54).

Sequences in the 3′ untranslated region in NOS3 and NOS2 may determine mRNA stability (Reference 55). RNA splicing contributes to altered mRNA and unique proteins in the various NOS isoforms (References 56 and 57). At the protein level, NOS function may be regulated in many ways: calmodulin binding, dimer formation (the enzyme requires dimerization for function), substrate (L-arginine) depletion, substrate recycling (L-citrulline to L-arginine), tetrahydrobiopterin (BH₄) availability, end product inhibition (NO interaction with NOS heme), phosphorylation, and subcellular localization. Important NOS co-factors include FAD, FMN, NADPH, tetrahydrobiopterin, and calmodulin-calcium. For NOS2, calmodulin is tightly bound to protein, making it relatively resistant to inhibition by calcium chelators. Activities of all NOS isoforms can be markedly influenced by levels of tetrahydrobiopterin-depleting cellular tetrahydrobiopterin by inhibitors of GTP cyclohydrolase 1, sepiapterin reductase, and dihydrofolate reductase reduces NOS activity (Reference 58). Cytokines and LPS can enhance tetrahydrobiopterin production.

The human NOS1 isoform of NOS is expressed from a very complex 240 kb locus at 12q24.2 composed of 19 exons (References 56, 57, 59 and 60). Although initially described in neural tissues, several tissues and cell types express NOS1. These include central and peripheral nervous tissue, muscle, and Leydig cells of the testis (References 56, 59 and 61). In the gastrointestinal tract, NOS1 acts as an important mediator of the non-adrenergic non-cholinergic inhibitory innervation of intestinal smooth muscle and as a neuromodulator within the enteric nervous system. Mice with disrupted NOS1 have gastromegaly and pyloric stenosis, and in humans with familial infantile pyloric stenosis NOS1, NOS1 shows disorded expression. These infants have a decrease in exon 1c mRNA in neurons innervating the pyloric sphincter, and a SNP in the NOS1 promoter for exon 1c is associated with increased risk for pyloric stenosis (Reference 62). NOS1 has never been reported in CLL cells or normal B cells, but some non-Hodgkin's lymphoma and multiple myeloma cells apparently do express NOS1 (Reference 36). There are at least 9 exon 1 variants (exon 1a-1i) that are used to initiate transcription in a tissue- and cell-specific manner through usage of alternative promoters (Reference 63). Some promoters act for NOS1 in neural tissues, while different ones for NOS1 in muscle or testicular tissue. NOS1 mRNA diversity is also generated by alternative splicing, and several variant NOS1 transcripts exist (References 64 and 65). These splice variants are functional and appear to respond differently to different stimuli and in different cell types. Finally, NOS1 is also regulated translationally, being influenced by an alternatively spliced exon in the 5′ untranslated region between exon 1 variants and a common exon 2 that contains the translational initiation codon (Reference 64). The amino terminal PDZ domain of NOS1 serves to localize the enzyme to critical regions of the cell. In neurons, PDZ binds postsynaptic protein (PSD)-95 and -93 proteins and thus co-localizes NOS1 and the NMDA receptor, while in myocytes, the PDZ domain co-localizes NOS1 and alpha-syntrophin. No one has studied this in B cells or CLL cells. PIN (protein inhibitor of nNOS) is a small protein of 89 amino acids initially described as a light chain subunit of dynein and as an inhibitor of NOS1. In vitro, PIN binds to a unique NOS1 domain encompassing amino acids 163-245. PIN inhibits NOS1 activity and blocks the formation of the active NOS1 dimer.

As noted above, NOS1 is considered a “constitutive” enzyme, with a basal transcription rate for a product whose activity is regulated by variations in calmodulin and cytoplasmic calcium concentrations. However, NOS1 mRNA transcription is also regulated by a physical factors and chemical and biological agents (Reference 66). Included are a variety of factors such as cytokines and insults such as ischemia/reperfusion injury (References 66-68). The NOS1 promoter contains candidate sequences for binding of AP-2, TEF-1/MCBF, CREB/ATF/c-Fos, NRF-1, Ets, NF-1, and certain NF-kB-like consensus sequences (Reference 60). Chesler and colleagues showed that IFN-y. could increase NOS1 expression in mouse neuroblastoma cells (Reference 67). They noted no change in mRNA steady state level and transcription rate, but there was increased translation of NOS1 protein from mRNA and increased stability of NOS1 protein. This indicates both a postranscriptional and posttranslational mechanism of cytokine modulation of NOS1 expression (Reference 67). This has not been examined in CLL cells.

Most NOS inhibitors bind to the oxygenase domain of NOS and interact with the guanidinium region of the arginine-binding site. Hibbs and colleagues described the importance of arginine in macrophage-mediated cytotoxicity, and demonstrated for the first time that arginine analogues such as N^{G}-monomethylarginine (NMMA) could inhibit cytotoxicity [a function they later described as being related to NO production (Reference 69)]. Since then, a variety of NOS inhibitors have been described (References 70 and 71). Arginine analogues that act as classic competitive inhibitors (e.g., L-thiocitrulline) bind to the oxygenase domain interacting with the guanidinium region of the arginine-binding site, are fully reversible, and are generally iso-form nonselective. “Slow on-slow off” arginine analogues (e.g., the S-alkyl-L-thiocitrullines) are not altered by NOS and also offer little isoform selectivity. Mechanism-based inhibitors [suicide inhibitors (e.g., NIO (N⁵-iminoethyl-L-ornithine))] offer the most isoform selectivity. Vinyl-L-NIO is an amidine analogue of this class that is markedly selective for NOS1. Likewise, L-NIL (L-N⁶-(1-Iminoethyl)-lysine) is very specific for NOS2. NOS oxidase inhibitors (e.g., diphenyleneiodonium which also inhibits NADPH oxidase) inhibits NO formation, and inhibitors of NOS dimer formation [e.g., various pyrimidineimidazoles (Reference 72)] blocks NO formation by NOS. NOS2-specific inhibitors have been targeted for use in a variety of conditions, most prominently septic shock and arthritis. NOS1-specific inhibitors have been targeted for use in psychiatric diseases such as depression and anxiety, and for neuro-degenerative diseases such as Alzheimer's disease and amyotrophic sclerosis (Reference 71). FIG. 1 shows structures of various amino acid NOS inhibitors (from Reference 70). It is noted that all structures are analogues of either L-arginine (the NOS substrate) or L-citrulline (the NOS product). (a) In the guanidino amino acids, a guanidium hydrogen of L-arginine is replaced by any of a variety of small substituents. (b) In the amidino amino acids, a guanidinium nitrogen atom of L-arginine is replaced by an alkyl, alkenyl or alkynyl group, whereas in (c) in the amino acid isothioureas a substituted sulfur replaces the guanidinium nitrogen atom. (d) The acetamidine lysine derivative resembles L-NIO, an amidino amino acid, but the carboxylate group is replaced by a vininal glycol. Like L-arginine, all of these amino acids, except L-NNA (N^{G}-Nitro-L-arginine) (a), have a strongly cationic sidechain. In contrast (e), L-citrulline and L-thiocitrulline are neutral amino acids.

Some NOS inhibitors have been used in research in humans. The nonselective inhibitor NMMA has been used in normals and in trials for septic shock (Reference 73) and for migrane headache (Reference 74). There were no major effects on cardiovascular, liver, or hematopoietic function. The prodrug for the NOS2-specific inhibitor L-NIL was administered orally to normal individuals and to those with asthma. This reduced exhaled NO with no effects on blood pressure, pulse, and respiratory function (Reference 75). There have been numerous preclinical studies in non-human animals of a variety of nonselective and selective inhibitors. Relative to this proposal, several NOS1-specific inhibitors have used studying their effects in animal models of amyotrophic lateral sclerosis, Parkinson's disease, Huntington's disease, Alzheimer's disease, depression, and anxiety (Reference 71). When trifluoromethyl phenylimidazole (TRIM) or 7-nitroimidazole (NI), we used at 50 mg/kg in mice, they were effective at reducing anxiety and depression. The only side effects were mild motor incoordination.

The simplified model in FIG. 2 depicts NO, NOS, caspases, apoptosis, and apoptosis inhibitors. It is noted that high level, exogenous NO (on the lower right of FIG. 2) generally leads to apoptosis and death of cells by a variety of mechanisms including direct membrane damage, and inhibition of ribonucleotide reductase, and inhibition of cellular generation of ATP by mitochondrial electron transport enzymes, aconitase, and GAPDH. Caspases (activated by a variety of signals) mediate apoptosis. Bcl-2 and NO can serve as apoptosis inhibitors. In contrast to exogenous, high level NO, endogenous, low level NO generally inhibits apoptosis, primarily by inhibiting caspases and modulating bcl-2 levels. Endogenous NO inactivates caspases by nitrosylation, and NO may also increase Bcl-2. NOS inhibitors and NO quenchers (“NOQ” in FIG. 2) facilitate apoptosis by reducing caspase inhibition. In FIG. 2, lines with a bar indicate inhibition, and dashed arrows indicate possible increase. CLL cells spontaneously overexpress NOS1 and NOS2, and NO produced in these cells inhibits apoptosis and death. We believe that this inhibition of apoptosis contributes to the leukemic process, and that NOS and NO are attractive treatment targets in this disease, which is a subject of the present invention.

In addition to CLL, NOS2 has been noted also in adult T cell leukemia-lymphoma cells from HTLV-1 (human T cell leukemia virus-1)-infected patients (Reference 76), in bone marrow cells from patients with myelodys-plastic syndrome (“preleukemia”) (Reference 77), and in hairy cell leukemia cells (Reference 78). Researchers have demonstrated NOS1 expression in non-Hodgkin's lymphoma and multiple myeloma tissues and cells (Reference 36). Thus, the importance of NO in leukemogenesis may extend beyond CLL to other forms of human leukemia.

### OBJECTS AND SUMMARY OF THE INVENTION

The principal object of the present invention is to provide a target for therapy in CLL.

An object of the present invention is to provide a NOS inhibitor for inducing apoptosis/cell death in CLL cells.

Another object of the present invention is to provide a NOS1-specific inhibitor for inducing apoptosis/cell death in CLL cells.

An additional object of the present invention is to provide a highly efficient agent for inducing apoptosis/cell death in CLL cells. A further object of the present invention is to demonstrate expression of NOS1 in CLL cells.

Yet a further object of the present invention is to demonstrate that NOS1 inhibitors can induce apoptosis/killing of CLL cells.

In summary, the present invention provides a target for therapeutic, diagnostic, and/or other uses in CLL.

One aspect of the present invention includes a method of inducing apoptosis or cell death in a cancer cell by inhibiting production of nitric oxide (NO) therein.

Another aspect of the present invention includes a method of inducing apoptosis or cell death in a leukemia cell by subjecting a leukemia cell to a NOS1-specific inhibitor.

Another aspect of the present invention includes a method of treating leukemia by administering to a subject in need thereof an effective amount of a NOS1-specific inhibitor for inhibiting the activity or expression of a nitric oxide synthase (NOS) in an affected cell.

### BRIEF DESCRIPTION OF THE DRAWINGS

One of the above and other objects, aspects, novel features and advantages of the present invention will become apparent from the following detailed description of the preferred embodiments(s) invention, as illustrated in the drawings, in which:

FIG. 1 illustrates chemical structures of selected NOS inhibitors;

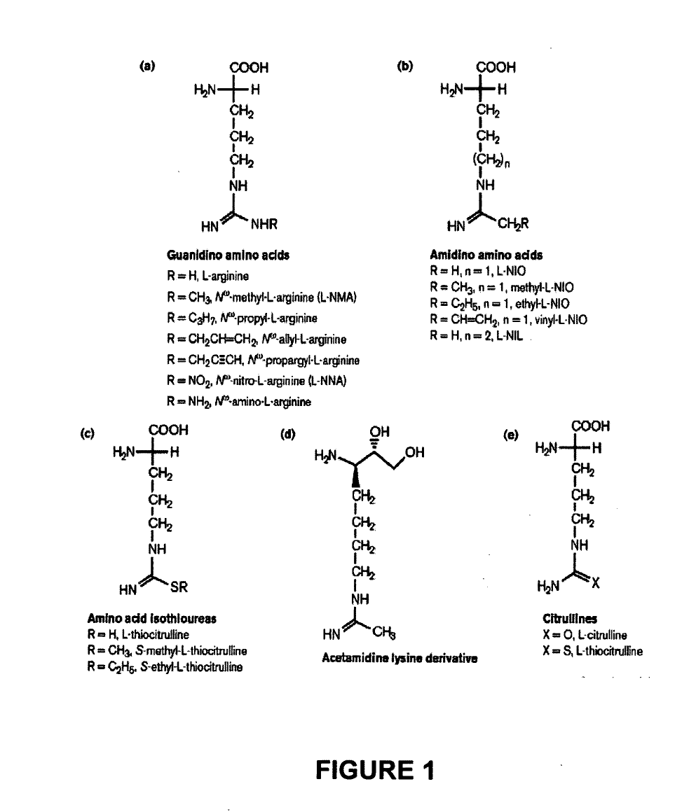

FIG. 2 illustrates interactions of nitric oxide and caspases in apoptosis. Abbreviations: L-arginine, L-arginine; NOS2, nitric oxide synthase type 2; NMMA, N^{G}-monomethylarginine; Ribo Reduc, ribonucleotide reductase; TNF-R1, tumor necrosis factor receptor 1; FADD, Fas-associated death domain; XRT, x-ray therapy; uv, ultraviolet light;

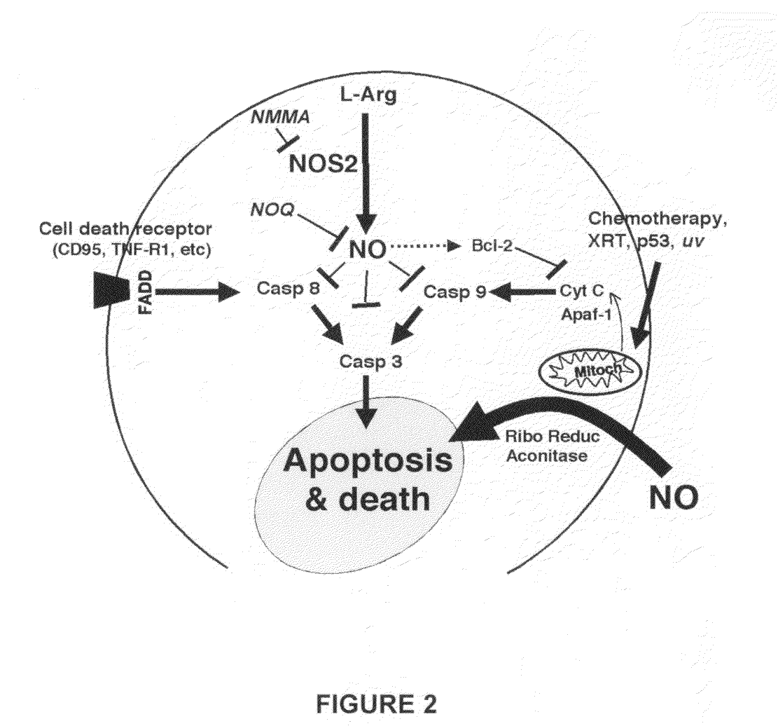

FIG. 3 is a graph illustrating influence of nitric oxide from sodium nitroprusside (SNP) on growth of colonies of erythroid (CFU-E) and granulocyte-macrophage (CFU-GM) cells in vitro;

FIG. 4 is a graph illustrating induction of apoptosis of CLL cells in vitro by the NO donors MAMA-NO ((Z)-1-{Nmethyl-N-[6-(N methylammoniohexyl)amino]}diazen-1-ium-1,2-diolate-NO), PAPA-NO ((Z)-1-[N-(3-ammoniopropyl)-N-(n-propyl)amino]-NO), and DETA-NO ((Z)-1-[N-(2-aminoethyl)-N-(2-ammonioethyl)amino]diazen-1-ium-1,2-diolate-NO);

FIG. 5 is a bar chart illustrating NOS enzymatic activity in lysates of blood mononuclear cells from normal individuals and those with CLL;

FIG. 6 are immunoblots for NOS2 and NOS3 of cell lysates of mononuclear cells from normal individuals (“NML”) and patients with CLL;

FIG. 7 illustrates real time reverse transcriptase-polymerase chain reaction (RT-PCR) quantitative analysis of NOS2 mRNA levels;

FIG. 8 is a graph illustrating quantitification of NOS2 mRNA from CLL cells treated for different times in vitro with nothing (“Control”), IL-4, or IFN-gamma;

FIG. 9A are photomicrographs of CLL cells examined by indirect immunofluorescence using specific antibodies directed against NOS1 or NOS2;

FIG. 9B displays immunoblots for NOS1 of cell lysates of B cells from normal individuals (“NI”) and patients with CLL; and

FIG. 10 is a graph illustrating cytotoxicity of NOS inhibitors for CLL cells in vitro. Curves show the mean±SEM percent cytotoxicity for inhibitors, with ED₅₀ displayed in the inset in micromolar concentrations (uM). The NOS1 inhibitor AR-17477 had the lowest ED₅₀, while the nonspecific NOS inhibitor NMMA had a very high ED₅₀. Abbreviations: AR-17477, N-(4-(2-((3-chlorophenylmethyl)amino)ethyl)phenyl)-2-thiophecarboxamidine dihydrochloride; ETPI, S-Ethyl-N-[4-(trifluoromethyl)phenyl]isothiourea.HCl; 7-NI, 7-nitroindazole; V-LNIO, N⁵-(1-Imino-3-butenyl)-L-ornithine; NMMA, N^{G}-monomethyl-L-arginine. From Levesque et al (Reference 106).

FIGS. 11A-E show correlations of NOS inhibitor IC50, Kd and cLogP values and the CLL cell ED50 of each compound. The NOS1 and NOS2 IC50's and Kd's for each NOS inhibitor were determined using purified recombinant full-length human NOS1 and NOS2. The cLogP value for each NOS inhibitor was determined using the CLOGP computer program (BioByte, Claremont, Calif.). Log normalized plots of the CLL cell ED50 for each compound vs. (A) NOS1 IC50, (B) NOS2 IC50, (C) NOS1 Kd, (D) NOS2 Kd and (E) cLogP for each compound are shown. Linear regression was used to examine the correlation between the IC50, Kd and cLogP values and ED50 values. Pearson's correlation coefficients (r²) and P-values for each plot are shown. Abbreviations: IC50, concentration at which there is 50% inhibition; Kd, dissociation constant; cLogP, calculated logarithm of the partition coefficient; ED50, concentration at which there is 50% inhibition.

### DETAILED DESCRIPTION OF THE PREFERRED EMBODIMENT(S) OF THE INVENTION

The present invention is based, in part, on one of the discoveries that CLL cells have NOS activity, produce NO, selectively express the NOS 2 isoform, and express high levels of NOS1 protein and NOS 1 mRNA.

While studying NO and macrophages progressed in the late 1980s, we hypothesized that NO produced in the BM (bone marrow) would be a modulator of normal and leukemic hematopoiesis. We noted that NO [delivered as NO-saturated buffer, or from the drugs nitroprusside, 6-morpholino-sydnonimine (SIN-1), or S-nitrosoacetylpenicillamine (SNAP)] potently inhibited the growth of HL-60 myeloblastic leukemia cells, and induced monocytic differentiation (Reference 1). This differentiation was associated with modulation of gene expression—NO treatment reduced expression of c-myc and c-myb mRNAs, and increased transcription of mRNA for IL-1 and TNF (as determined by run-on experiments). Our work was the first to show that NO could modulate gene expression in any cell type. The differentiated cells were vacuolated, and had increased expression of nonspecific esterase, CD11b, and CD14.

We then analyzed freshly-isolated leukemia cells from 20 patients with ANLL (acute nonlymphocytic leukemia) for their responses to NO in vitro (Reference 41). It was important to do this, since cells of leukemia cell lines may not accurately reflect the actions of cells in vivo. Freshly-isolated cells all responded to NO treatment (decreased growth or induced monocytic differentiation), but overall their responses were less consistent than we noted with the more uniform cell line HL-60. Cells of monocytic phenotype ANLL (M4 and M5) were the most responsive to NO treatment.

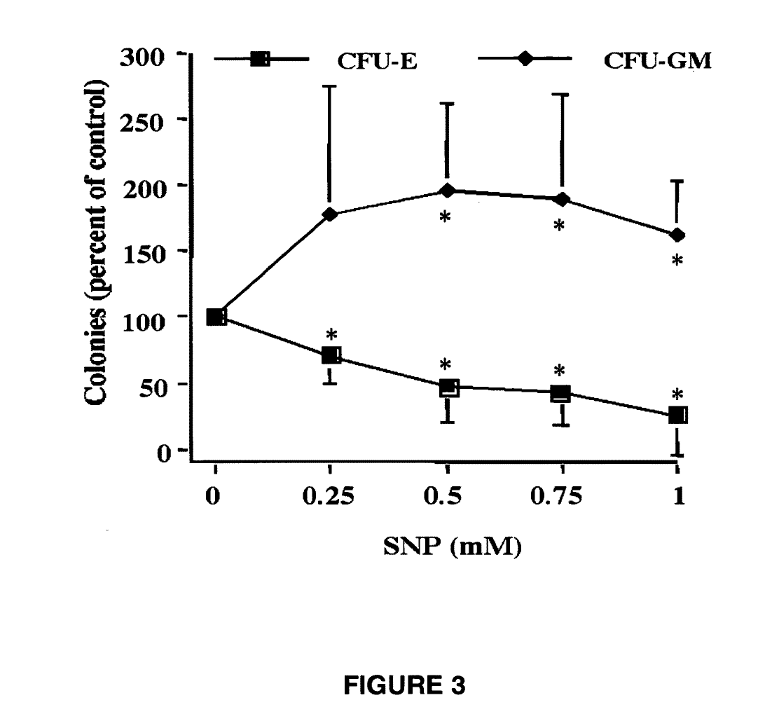

The effects of NO on the growth and differentiation of normal human BM cells were analyzed (Reference 85). We felt that normal hematopoietic cells, like malignant hematopoietic cells, would be affected by NO. NO delivered from the drugs nitroprusside, SIN-1 (6-morpholino-sydnonimine), or SNAP inhibited development of marrow colonies when cells were cultured in methylcellulose with erythropoietin and colony stimulating factors. NO reduced formation of BFU-E (burst forming unit-erythroid), CFU-E, CFU-GM (colony forming unit-granulocyte/macrophage), and CFU-M (colony forming unit-macrophage). Using purified CD34+ cells, we showed that the NO most likely affected the hematopoietic precursor cells and not adherent cells (some of the “stromal” BM cells). When using isolated CD34+ cells, both erythroid and myeloid (moreso for erythroid) colonies were inhibited by SNAP, while SNP inhibited BFU-E and increased CFU-GM (FIG. 3). In retrospect, we think that the increase in CFU-GM by NO from SNP may be due to alterations in apoptosis secondary to changes in bcl-2 or caspases. None of the noted inhibitions were related to cGMP (cyclic-guanosine monophosphate).

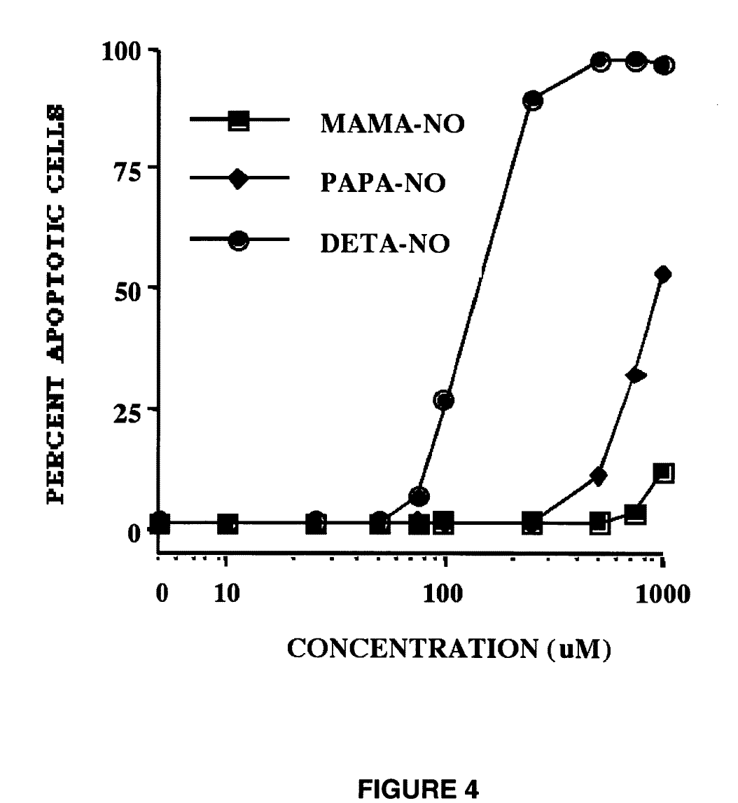

To more closely examine the mechanism of toxicity for myeloid leukemia cells, we did work to determine whether the rate of NO delivery affected its growth inhibition of acute nonlymphocytic leukemia cells. We also wanted to determine whether the NO inhibition of cell growth is associated induction of apoptosis. We treated HL-60 and U937 cells with three compounds that generate the same amount of NO but different rates. FIG. 4 shows the degree of apoptosis induced in HL-60 cells after treatment with the diazeniumdiolates MAMA-NO, PAPA-NO and DETA-NO(NO-donating agents with have half-lives of NO delivery of 2 and 30, and 1200 min), respectively. The compound with the longest half time of NO delivery (DETA-NO) was the most potent inhibitor of leukemia cell and colony growth. Furthermore, NO-induced growth inhibition was associated with apoptosis in a rate and concentration-dependent fashion (Reference 39).

We next wanted to examine CLL cells. These malignant cells differ in many ways from ANLL and normal hematopoietic. In addition to their different lineage, they are unique in that they have a very low growth factor, exist primary in the G₀ phase of the cell cycle, and have defective apoptosis. In a fashion comparable to what we had done with ANLL cells, we tested the effects of acute addition of exogenous NO donors on the freshly-isolated CLL cells (Reference 40). CLL cell apoptosis and death were induced by the pure NO donors DETA-NO (ID50 188 uM), PAPA-NO (ID50 850 uM), and MAMA-NO (ID50 1658 uM). The agents' potencies were comparable to those for ANLL, with the cytotoxic effect being inversely related to the NO release rates of the donors (Reference 39). DETA alone (without NO in the molecule), or NO-depleted DETA-NO had no effects. The ID50 for fludarabine was 2 uM. DETA-NO acted synergistically with fludarabine to kill the cells. NO also synergized with the ara-guanosine prodrug 2-amino-9-β-D-6-methoxy araguanine (also called 506U78). However, the NO-drug interactions were restricted; DETA-NO did not enhance the activity of several other agents (5-fluorouracil, gemcitabine, doxorubicin, chlorambucil, or the CPT-11 metabolite SN-38).

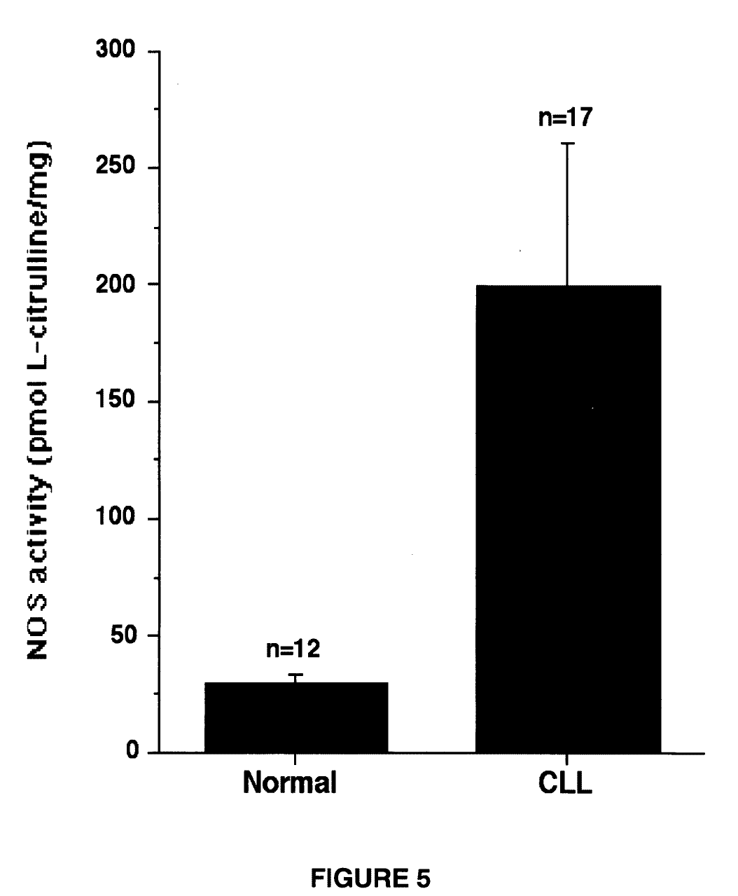

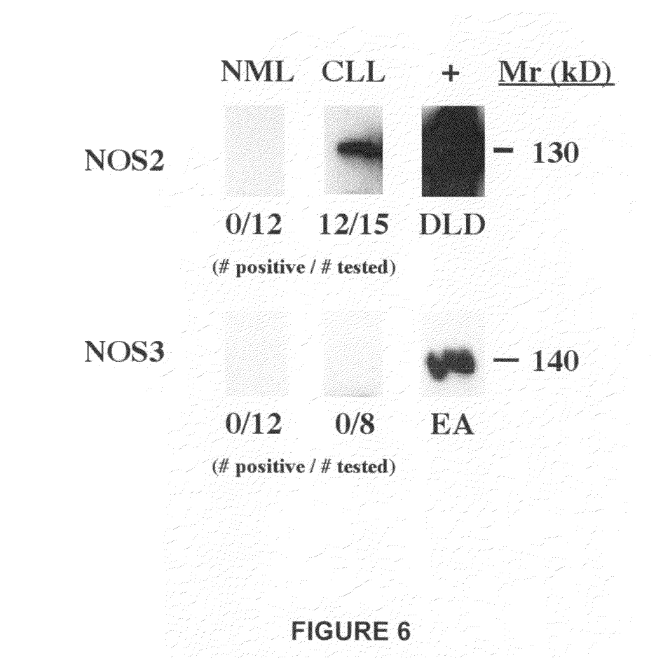

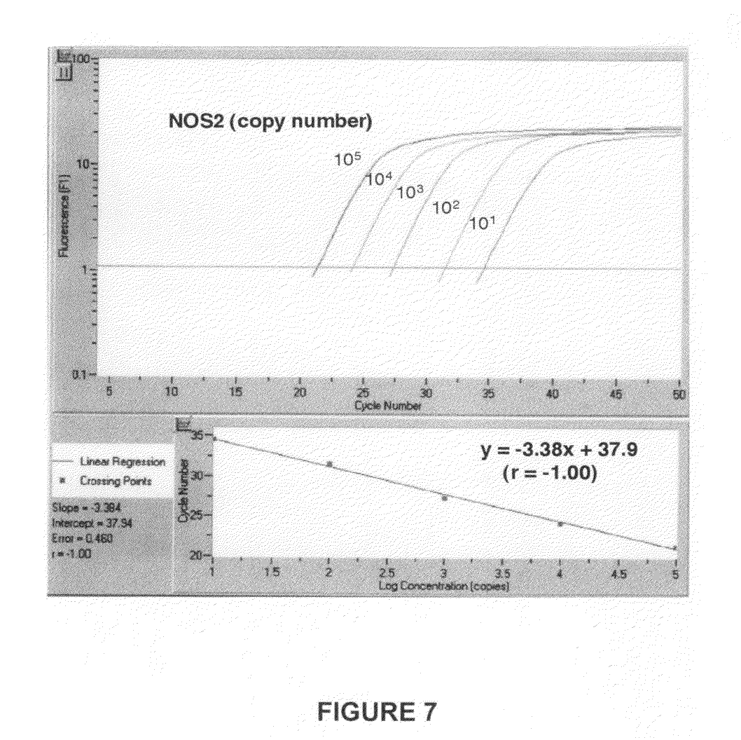

We considered the possibility that endogenously produced NO might affect CLL cell survival. Although most think of mononuclear phagocytes when they think of NO, normal T and B lymphocytes have been reported to contain NOS2 and NOS3 (see above). CD5+ B lymphocytes share many features with macrophages (Reference 12). Thus, we postulated that the CD5+ B lymphocytes of CLL would express functional NOS2 (Reference 9). Our results were published at about the same time as those of another group that reported expression of NOS2 by CLL cells (Reference 11). We found that CLL cells have NOS activity, produce NO, and selectively express the NOS2 isoform. The patients studied all had typical CLL, with CD5+, CD19+ B cell disease; some had had no treatment, while others had received chemotherapy. In all patients, the WBC was more than 20,000/ul, and they had not received any chemotherapy within 4 weeks of phlebotomy. We found increased NOS enzyme activity (as measured by conversion of 14-C-L-arginine to 14-C-L-citrulline) in CLL cell samples (n=17 from 13 patients) compared to blood MNC from normal individuals (n=12 from 12 subjects) [FIG. 5 (mean±SEM); p<0.02 (Reference 9)]. Immunoblot analysis (FIG. 6) detected NOS2 in most of the CLL samples. In contrast, NOS2 and NOS3 were not detected by immunoblot analysis of purified B cell from normal controls (N=12). With RT-PCR, we found NOS2 (but not NOS3 noted mRNA in cells from 12/13 CLL patients studied, while NOS2 and NOS3 mRNA were absent in normal controls. The control for cellular mRNA was GAPDH. DLD represents the human colon cancer cell line treated with IFN-α, TNF, & IL-1 (+ for NOS2 and NOS3). EA is a human endothelial cell-epithelial cell hybrid line (+ for NOS3). Using real-time RT-PCR (see FIG. 7 for example, of a standard curve using this technique), we have been able to quantify the NOS2 mRNA (Reference 10).

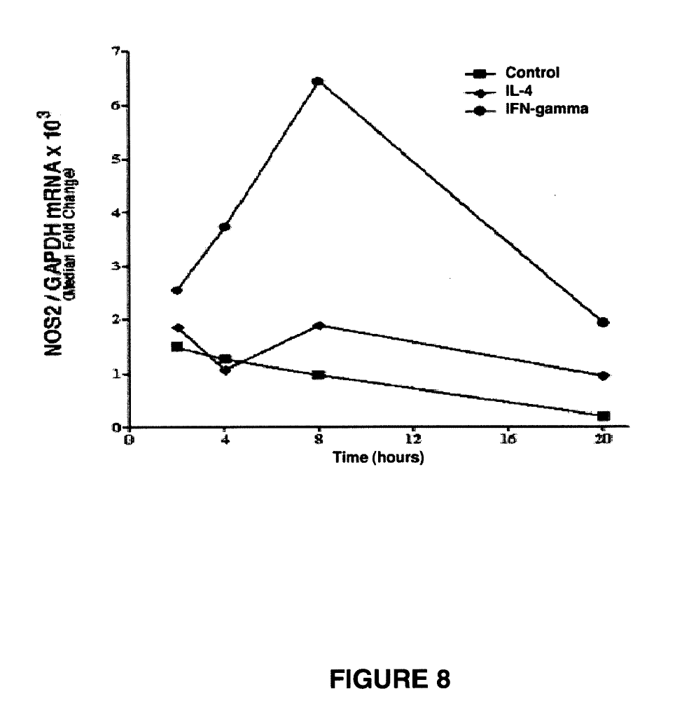

We investigated the effects of different cytokines and growth factors on the viability of CLL cells in vitro, NOS2 expression, and spontaneous and NOS-inhibitor induced cell death (Reference 10). Culture of cells with IL-4 or IFN-γ (but not TNF-α, IL-2, IL-6, IL-8, G-CSF, nerve growth factor, or IFN-α) increased NO production. By quantitative RT-PCR, IL-4 increased NOS2 mRNA (FIG. 8). Also, 5 of 5 patients' CLL cells had increases in NOS2 protein (immunoblot) after in vitro treatment with IL-4. Apoptosis (TUNEL assay) was induced by NMMA treatment of the cells, and incubating cells with IL-4 or IFN-y reduced apoptosis. This suggested that cytokine-induced NO prevents NMMA-induced apoptosis. Since IL-4 and IFN-gamma induce NOS2 and modulate CD38 expression in CLL cells in vitro, we sought to determine if CLL patients had elevated levels of these cytokines and if the levels related to CD38 expression by the leukemia cells (Reference 87). Our study of 170 serum samples from 64 different patients showed that serum IL-4 levels were significantly elevated in CLL patients, and that there was an association of IL-4 levels with the absence of CD38 expression and increased NOS2 expression.

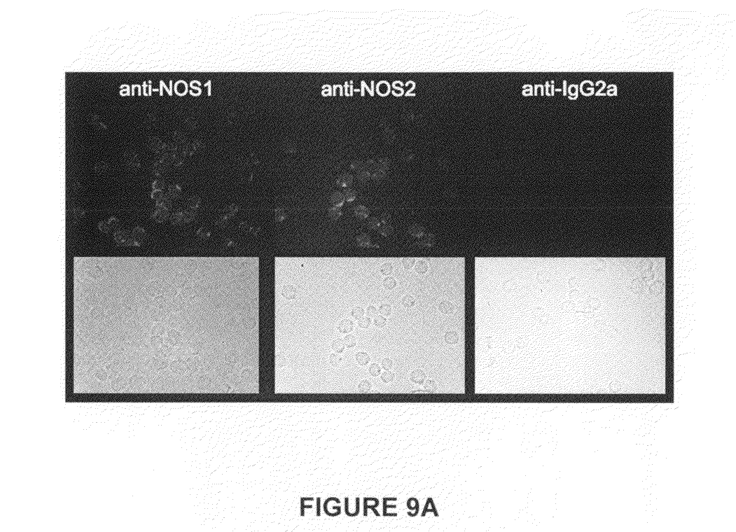

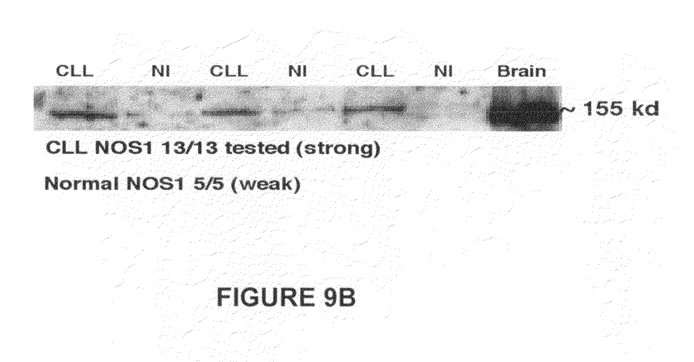

We noted presence of NOS1 and NOS2 CLL cells in 11 of 11 cases in which we tested using specific anti-NOS1 and anti-NOS2 antibodies in indirect immunofluorescence studies for these proteins (FIG. 9A) (Reference 106). We also noted NOS1 protein by immunoblot (FIG. 9B) in 41 of 43 CLL samples tested, and NOS1 mRNA by RT-PCR in 56 of 149 (38%) of CLL samples. The relatively low positivity rate for NOS1 mRNA compared to NOS1 protein by indirect immunofluorescence and immunoblot is likely caused by a short life of mRNA in the cells compared to that for the NOS1 protein. NOS1 protein was noted in 5 of 5 PBMC samples from normal individuals, but only with low intensity staining (approximately to 5 to 10% of the density of the CLL cells).

We worked to optimize the culture of CLL cells (Reference 86). When we cultured CLL cells in DMEM or RPMI-1640 media with fetal bovine or human serum, approximately 20 to 30% of the cells died within 24 to 72 hours. However, when cells were cultured in serum-free media (especially, serum-free “Hybridoma-SFM” medium, GIBCO (Grand Island Biological Company)), there was 99±7% (SEM) (n=28) viability at 72 hours with little apoptosis. Other media tested include serum-free DMEM and RPMI-1640, “AIM-V (Invitrogen located in Carlsbad, Calif.),” and Iscove's modified DMEM. These were all tested with and without albumin, fetal bovine serum, human serum, glutamine, insulin, transferrin, selenium, and mercaptoethanol. Serum-free Hybridoma-SFM was clearly the best.

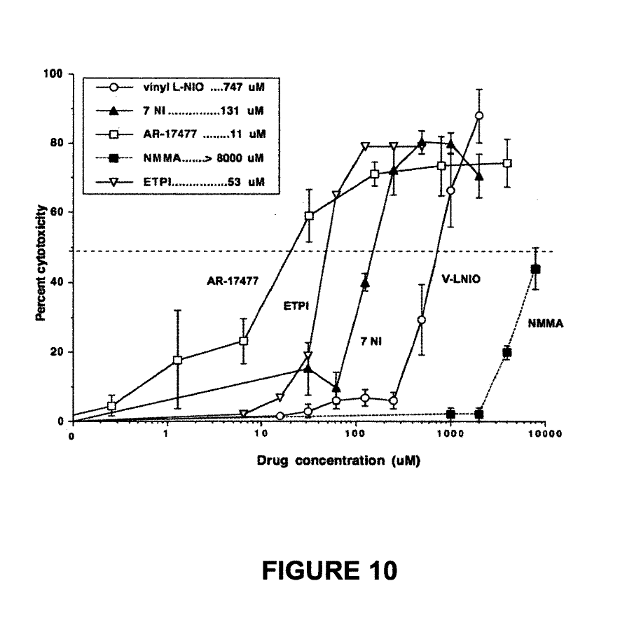

When CLL cells were cultured with various NOS inhibitors, there was dose-dependent killing of cells; this was apparent as early as 12 to 14 hours. The cells were very refractile and pyknotic by phase contrast microscopy. Cytotoxicity was of high level (up to 100% dead cells). Toxicity could be detected by disappearance of cells from the culture (reduction in overall cell number), uptake of trypan blue, propidium iodide uptake (flow cytometry), MTS (4,5-dimethylthiazol-2-yl)-5-(3-carboxymethoxyphenyl)-2-(4-sulfophenyl)-2H-tetrazolium, inner salt) assay (cellular respiration), and several parameters of apoptosis [annexin V assay, DNA content (<in), and TUNEL assay]. FIG. 10 displays the cytotoxicity of some of the NO inhibitors for CLL cells. In the present invention studying cytotoxicity of NOS inhibitors for CLL cells, we did the following number of experiments: vL-NIO, n=7; 7-NI, n=6, ARL-17477, n=6; NMMA, n=6.

Sensitivity to NOS inhibitors varied somewhat among patients. Generally, cells from most essentially all patients were sensitive to NOS inhibitor-induced death. We screened several isoform nonspecific and specific NOS inhibitors (see Table 1 for list of inhibitors tested and some of their characteristics) and NO quenchers/scavengers, for example hydroxocobalamin (Reference 105) and carboxy-PTIO. The non-specific NOS inhibitors and NOS2-specific inhibitors either did not induce CLL cell death or induced CLL cell death with EC₅₀ values (average concentration of compound that induced 50% CLL cell death) over 2000 uM. In contrast, NOS1-specific inhibitors induced CLL cell death at much lower concentrations (Table 2) (Reference 106).

TABLE 1
List of NOS inhibitors tested, their abbreviations, and their NOS specificities
NOS inhibitor characteristics

| NOS inhibitor | Full chemical name^{a} | Specificity |
|---|---|---|
| L-NAME | N^{G}-Nitro-L-arginine-methyl ester HCl | NOS |
| L-NMMA | N^{G}-Monomethyl-L-arginine monoacetate | NOS |
| S-Ethyl ITU | S-Ethylisothiourea HBr | NOS |
| 7-NI | 7-Nitroindazole | NOS1 |
| 3-Bromo-7-NI | 3-Bromo-7-nitroindazole | NOS1 |
| Vinyl-L-NIO | N⁵-(1-Imino-3-butenyl)-L-ornithine | NOS1 |
| AR-R17477 | N-(4-(2-((3-Chlorophenylmethyl)amino)ethyl)phenyl)- | NOS1 |
| | 2-thiophecarboxamidine dihydrochloride | |
| NAAANG | (4S)-N-(4-Amino-5[aminoethyl]aminopentyl)-N′-nitroguanidine 3TFA | NOS1 |
| ETPI | S-Ethyl-N-[4-(trifluoromethyl)phenyl]isothiourea HCl | NOS1 |
| TRIM | 1-(2-Trifluoromethylphenyl)imidazole | NOS1 |
| N-Propyl-L-arginine | N⁵-[Imino(propylamino)methyl]-L-ornithine | NOS1 |
| L-NNA | N^{G}-Nitro-L-arginine | NOS1 |
| 1400W | N-(3-(Aminomethyl)benzyl)acetamidine 2HCl | NOS2 |
| L-NIL | L-N⁶-(1-Iminoethyl)-lysine | NOS2 |
| AMT | 2-Amino-5,6-dihydro-6-methyl-4H-1.3-thiazine HCl | NOS2 |
| 1,3 PB-ITU | S,S′-(1,3-Phenylene-bis(1,2-ethanediyl))bis-isothiourea 2HBr | NOS2 |
| 1,4 PB-ITU | S,S′-(1,4-Phenylene-bis(1,2-ethanediyl))bis-isothiourea 2HBr | NOS2 |
| GW274150 | (S)-2-Amino-(1-iminoethylamino)-5-thioheptanoic acid | NOS2 |
| GED | Guanidinoethyldisulfide 2H₂CO₃ | NOS2 |
| MEG | Mercaptoethylguanidine sodium succinate | NOS2 |
| S-Methyl ITU | S-methylisothiourea sulfate | NOS2 |
| S-Aminopropyl ITU | S-(3-Aminopropyl)isothiourea 2HBr | NOS2 |

In general, the lower the Kd for inhibiting recombinant purified human NOS1, the more likely the compound was to kill CLL cells. NO binders/quenchers [(carboxy-PTIO [2-(4-carboxyphenyl)-4,4,5,5-tetramethylimidazoline-1-oxy]-3-oxide and hydroxycobalamin] were not cytotoxic for CLL cells.

TABLE 2
ED50 values for NOS inhibitors relative to cytotoxicity for CLL cells
CLL cell ED₅₀ values for NOS inhibitors

| NOS inhibitor^{a} | Specificity^{b} | Number tested^{c,d} | Concentration range (μM) | Geometric mean ED₅₀^{e} | Mean ED₅₀ ± S.E.M.^{f} |
|---|---|---|---|---|---|
| L-NAME | NOS | 4 | 125-8000 | 6916 | 7225 ± 1304 |
| L-NMMA | NOS | 18 | 16-8000 | 8659 | 11944 ± 3746^{g} |
| S-Ethyl ITU | NOS | 7 | 31-4000 | 4035 | 4487 ± 812^{g} |
| 7-NI | NOS1 | 20 | 8-2000 | 161 | 200 ± 29 |
| 3-Bromo-7-NI | NOS1 | 5 | 8-500 | 220 | 225 ± 20 |
| Vinyl-L-NIO | NOS1 | 5 | 16-2000 | 905 | 1190 ± 456 |
| AR-R17477 | NOS1 | 12 | 0.3-4000 | 6.4 | 7.0 ± 0.7 |
| NAAANG | NOS1 | 3 | 16-4000 | 3636 | 5231 ± 3197 |
| ETPI | NOS1 | 6 | 8-500 | 119 | 129 ± 20 |
| TRIM | NOS1 | 3 | 8-500 | 411 | 421 ± 61 |
| N-Propyl-L-arginine | NOS1 | 5 | 31-2000 | 12038 | 17229 ± 6414^{g} |
| L-NNA | NOS1 | 5 | 31-2000 | 7183 | 55666 ± 47825^{g} |
| 1400W | NOS2 | 11 | 16-8000 | 5893 | 8244 ± 1885 |
| L-NIL | NOS2 | 17 | 16-8000 | 8617 | 12058 ± 1824^{g} |
| AMT | NOS2 | 4 | 8-500 | 1081 | 1403 ± 628^{g} |
| 1,3 PB-ITU | NOS2 | 4 | 23-500 | 289 | 293 ± 26.5 |
| 1,4 PB-ITU | NOS2 | 4 | 23-500 | 313 | 321 ± 38.9 |
| GW274150 | NOS2 | 6 | 4-500 | 2156 | 2600 ± 773^{g} |
| GED | NOS2 | 4 | 23-500 | 290 | 317 ± 77 |
| MEG | NOS2 | 3 | 8-500 | 174 | 181 ± 36.4 |
| S-Methyl ITU | NOS2 | 7 | 31-4000 | 2793 | 3077 ± 423 |
| S-Aminopropyl ITU | NOS2 | 3 | 8-500 | 94 | 112 ± 45.5 |

^{a}Abbreviated name for NOS inhibitors; full chemical names for each inhibitor are listed in Table 1.
^{b}NOS: inhibits all NOS isoforms approximately equally; NOS1, NOS2: relative specificity for NOS1 and NOS2 isoforms, respectively.
^{c}MTS assay results for all compounds except vinyl-L-NIO (determined by PI exclusion assay).
^{d}Number of CLL samples tested with each compound. For each compound, CLL cell samples were from different subjects.
^{e}ED₅₀ calculated as described in Section 2.
^{f}S.E.M.: standard error of mean.
^{g}Samples with means greater than the upper limits of the tested concentration range represent estimates based on extrapolation of toxicity data at lower concentrations.

The pattern of CLL cell toxicity in Table 2 and FIG. 10 did not explain why some NOS inhibitors were more potent inducers of CLL cell death. Therefore, we examined other factors related to the NOS inhibitors that might explain the pattern of CLL cell toxicity observed in Table 2. We examined the specificity and potency of the NOS inhibitors by determining the concentration of each compound that inhibited 50% of the enzyme activity (IC50) of purified recombinant NOS1 and NOS2. We determined the dissociation constant for NOS inhibitor binding (Kd) to purified recombinant and NOS2. Finally, we used a computer algorithm to estimate each NOS inhibitor's hydrophobicity (cLogP) (Reference 106). The results of the IC50, Kd and cLogP determinations for each NOS inhibitor are summarized below in Table 3.

TABLE 3
IC50, Kd, and cLogP values for NOS inhibitors
IC₅₀. K_{d} and cLogP values for NOS inhibitors

| NOS inhibitor^{a} | Specificity^{b} | NOS1 IC₅₀^{c} | NOS2 IC₅₀^{d} | NOS1 K_{d}^{e} | NOS2 K_{d}^{f} | cLogP^{g} |
|---|---|---|---|---|---|---|
| L-NAME | NOS | 0.89 ± 0.06 | 17 ± 1.5 | 0.5 | 0.6 | −3.4 |
| L-NMMA | NOS | 6.4 ± 1.6 | 8.7 ± 1.5 | 0.5 | 0.7 | −4.0 |
| S-Ethyl ITU | NOS | 7.7 ± 1.8 | 1200 ± 290 | ND^{h} | ND | UNⁱ |
| 7-NI | NOS1 | 72 ± 16 | 45 ± 5.9 | 0.1 | ND | 1.7 |
| 3-Bromo-7-NI | NOS1 | ND | ND | ND | ND | 2.7 |
| Vinyl-L-NIO | NOS1 | ND | ND | 0.1 | 0.5 | −1.5 |
| AR-R17477 | NOS1 | 0.28 ± 0.08 | 0.92 ± 0.06 | 0.03 | 0.4 | 4.8 |
| NAAANG | NOS1 | ND | ND | ND | ND | −5.7 |
| ETPI | NOS1 | 7.8 ± 2.5 | 1500 ± 240 | 0.1 | 0.5 | 4.5 |
| TRIM | NOS1 | ND | ND | ND | ND | 2.8 |
| N-Propyl-L-arginine | NOS1 | 11.5 ± 3.3 | 146 ± 36 | 0.7 | 7 | UN |
| L-NNA | NOS1 | 0.34 ± 0.08 | 6.1 ± 1.9 | 0.5 | 0.9 | −3.5 |
| 1400W | NOS2 | 20 ± 0.3 | 0.4 ± 0.1 | 1.5 | 0.1 | 0 |
| L-NIL | NOS2 | 47 ± 1.2 | 2.1 ± 0.0 | 0.7 | 0.5 | −2.9 |
| AMT | NOS2 | 0.092 ± 0.002 | 0.054 ± 0.001 | ND | ND | 1.6 |
| 1,3 PB-ITU | NOS2 | 4.7 ± 0.8 | 1.6 ± 0.3 | ND | ND | UN |
| 1,4 PB-ITU | NOS2 | ND | ND | ND | ND | UN |
| GW274150 | NOS2 | 140 ± 15 | 1.8 ± 0.3 | ND | ND | UN |
| GED | NOS2 | 280 ± 80 | 50 ± 9.5 | ND | ND | −1.34 |
| MEG | NOS2 | ND | ND | ND | ND | −0.87 |
| S-Methyl ITU | NOS2 | ND | ND | ND | ND | UN |
| S-Aminopropyl ITU | NOS2 | 3.8 ± 0.5 | 1.9 ± 0.2 | ND | ND | UN |

^{a}Abbreviated name for NOS inhibitors: full chemical names for each inhibitor are listed in Table 5.
^{b}NOS: inhibits all NOS isoforms equally; NOS1, NOS2: relative specificity for NOS1 and NOS2 isoforms, respectively.
^{c}Mean concentration (μM) of each compound that inhibited 50% of purified human NOS1 enzyme activity ± standard error of mean concentration (SEM). N = 3 for each compound.
^{d}Mean concentration (μM) of each compound that inhibited 50% of purified human NOS2 enzyme activity ± standard error of mean (SEM). N = 3 for each compound.
^{e}K_{d} (μM) for each compound performed using purified recombinant rat NOS1 oxygenase domain.
^{f}K_{d} (μM) for each compound performed using purified recombinant human NOS2 oxygenase domain.
^{g}Partitioning coefficient of compound in octane vs. water estimated using the CLOGP program (BioByte).
^{h}Not determined.
ⁱUnable (UN) to estimate cLogP for these compounds using the CLOGP program (BioByte).

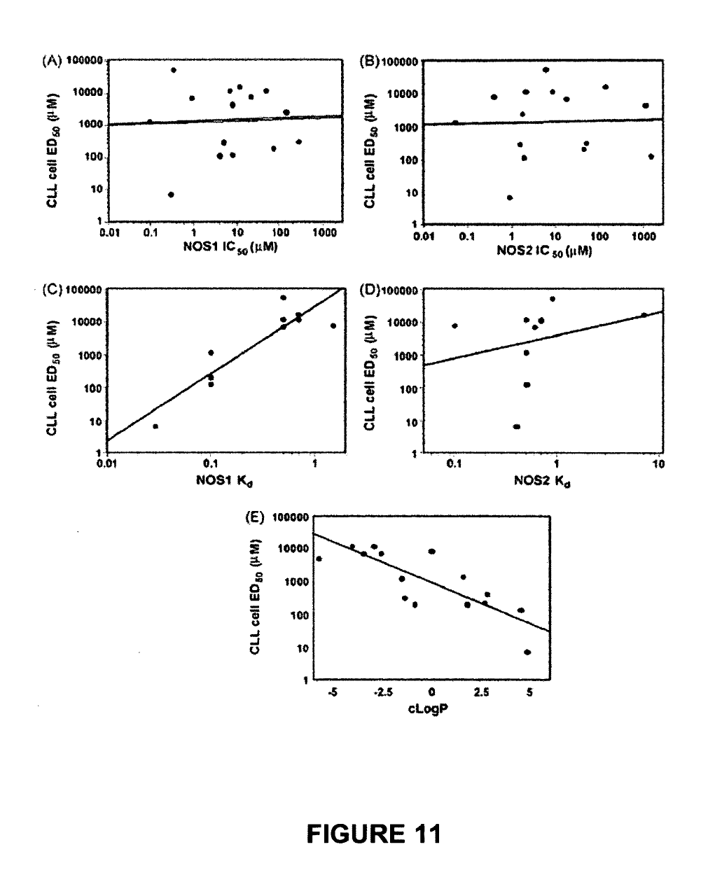

We determined whether the IC50, Kd and cLogP (FIG. 11) for each NOS inhibitor were associated with the CLL cell ED50 of each NOS inhibitor using linear regression (Reference 106). There was no correlation of the NOS1 and NOS2 IC50 values with CLL cell ED50 values (FIGS. 11A and B). There was an excellent correlation between the NOS1 Kd (but not NOS2 Kd) for each NOS inhibitor and the CLL cellED50 of each compound (r²=0.81, P=0.0004 for NOS1 Kd) (FIGS. 11C and D). There was also an excellent correlation between the cLogP and CLL cell ED50 of each NOS inhibitor (r²=0.64, P=0.0004) (FIG. 11E). There was a correlation between the NOS1 Kd and cLogP for each compound (r²=0.52, P=0.0284), and in a multiple linear regression model, the Kd and cLogP were independently associated with the CLL cell ED50 for each NOS inhibitor (Table 4) (Reference 106).

TABLE 4
Multilinear regression analysis of factors associated
with CLL ED50 values for NOS inhibitors^{a}

| Factor | Effect | Estimate | r² | P-value |
|---|---|---|---|---|
| cLogP | Inverse | −0.1990 | 0.8256 | 0.0073 |
| NOS1 K_{d}^{b} | Direct | 1.136 | 0.9453^{c} | 0.0111 |

^{a}P = 0.0002 (analysis of variance)| for overall multiple linear regression model.
^{b}All continuous variables were log normalized prior to analysis.
^{c}Cumulative r² values, i.e. r² value for NOS1 K_{d} includes effects of NOS1 K_{d} and cLogP.

Taken together, this analysis suggested that compounds with the highest specificity for NOS1 and those that were the most hydrophobic were likely to be the most toxic for CLL cells.

We determined whether the NOS1 specific inhibitor AR-R17477 induced CLL cell apoptosis using Annexin V and caspase-3 enzyme activity. We tested the AR-R17477 compound at various concentrations, and the percentage of apoptotic CLL cells and caspase-3 enzyme activity were determined at various time points following addition of AR-R17477 to CLL cell cultures (Reference 106). CLL cell caspase-3 enzyme activity increased after CLL cell co-culture with AR-R17477 for 4 to 19 h. Annexin V binding to CLL cells increased after CLL cell co-culture for 2-6 h. In CLL cells cultured with various concentrations of AR-R17477 for 4 h, there was a dose-dependent increase in caspase-3 enzyme activity (Table 5) (Reference 106).

TABLE 5
CLL cell caspase-3 enzyme activity following 4 h
culture with various concentrations of AR-R17477

| AR-R17477 | Mean caspase-3 | | |
|---|---|---|---|
| concentration (μM) | activity^{a} | S.E.M. | P-value^{b} |
| 0 | 3952 | 635 | — |
| 5 | 9741 | 4426 | 0.3710 |
| 10 | 7474 | 552 | 0.0871 |
| 20 | 8074 | 357 | 0.0114 |

^{a}N = 3; arbitrary fluorescence units.
^{b}P-value comparison using matched pairs t-test of mean caspase-3 activity compared with mean caspase-3 activity for 0 μM concentration of AR-
R17477.

Taken together, these results demonstrated that the NOS1 specific inhibitor AR-R17477 induced CLL cell apoptosis and caspase-3 enzyme activity.

We determined whether freshly-isolated and cultured CLL cells produced detectable NO and whether cytokine treatment of CLL cells augmented NO production. We cocultured B-CLL cells with IL-2, IL-4, IL-6, IL-8, IFN-gamma, IFN-alpha, NGF (nerve growth factor), or G-CSF (granulocyte-stimulating factor) and measured nitrite and nitrate concentrations in the culture media by several different techniques including the Griess reaction and by the sensitive nitric oxide analyzer using the chemiluminescence technique (NOA from Sievers) (Reference 10). None of these cytokines induced detectable NO production, even though IL-4 and IFN-gamma increased NOS2 protein, and IL-4 increased NOS2 mRNA expression. We also cocultured the cells with IL-4, IFN-gamma or IFN-alpha in the presence of increased amounts of arginine (1 to 10 mM) and/or sepiapterin (100 uM) for 1, 3 or 5 days. None of these culture conditions induced NO production. This was somewhat surprising, but very reproducible. Other investigators have noted comparable difficulty demonstrating NO production in vitro, even when NOS inhibitors produced dramatic biologic consequences. This is especially true when NOS1 is functioning, since it results in biologically significant changes despite producing only nM amounts of NO.

In our judgement, there are no suitable human CLL cell lines that are comparable to usual CLL cells. They are frequently EB virus infected, they proliferate, and most are CD5 negative. Thus, all of our CLL experiments were done with freshly-isolated cells from patients with CLL. In the last 2-3 years, we have collected blood from 114 different patients with CLL, and in most we have drawn blood more than once. We have now over 2718 unused cell pellets (10 to 100 million cells per pellet) and 717 separate plasma samples from these subjects. We have been doing several types of assays. We have done immunophenotyping (CD3, CD19, CD20, CD23, CD14, and CD38) on all samples isolated from the subjects. In many, we have done detailed studies of cell survival in vitro with or without various drugs, apoptosis assays, and immunoblots for various antigens. Recently, we have been done immunoblots for Zap70, and have successfully developed a sensitive flow cytometric assay for intracellular Zap70. In 90 patients, we tediously sequenced immunoglobulin heavy and light chains to determine their somatic mutational status. We have detailed, finalized information on 74 of these 90 regarding Ig H chain mutation status, CD38 positivity, Rai and Binet stage, lymphocyte doubling time, and diagnosis-to-treatment time (Reference 90). In brief (Table 6 below), our results to date show a correlation between CD38 negativity and presentation of mutated Ig H chains (p=0.0008), more Ig H chain mutation in Rai stages 0, 1, & 2 (compared to 3 & 4, p=0.03), higher CD38 positivity in Rai stages 0, 1, & 2 (compared to 3 & 4), lower lymphocyte doubling time in those with unmutated Ig H chains (p=0.008), and lower doubling time in those with CD38+ CLL cells. Patients with unmutated Ig H chains had a shorter time from diagnosis to treatment. Likewise, those with CD38+ cells had a shorter time from diagnosis to treatment. These results in general correspond to those published by other investigators. CD38, Ig H mutation status, diagnosis-to-treatment time, and lymphocyte doubling time did not significantly correlate with CLL cell NOS enzyme activity. These collected cells and plasma/sera and the clinical and laboratory data will be useful in our future planned studies.

TABLE 6
CLL Characteristics According to Ig H Chain Mutation Status

| | | | | | | | | Doub | DxTo | | |
|---|---|---|---|---|---|---|---|---|---|---|---|
| | # | Rai | Binet A | Binet B | Binet C | CD38− | CD38+ | Time | Rx | Female | Male |
| H unmutated | 23 | 2.7 | 5(29%) | 2(12%) | 10(59%) | 3(14%) | 14(82%) | 1546 ± 108 | 1284 ± 210 | 29% | 71% |
| H mutated | 51 | 1.5 | 20(56%) | 5(14%) | 11(31%) | 24(67%) | 12(33%) | 4779 ± 308 | 2643 ± 130 | 22% | 78% |

A total of 74 patients have had Ig H chain sequencing. Unmutated signified less than 2% of the bases are different than germline sequence. The number of subjects analyzed to-date for the various parameters varies from 38 to 74.

As part of our detailed analyses, we have been investigating individually sorted cells to determine certain parameters of the isolated leukemia cells. We have started to investigate Ig H and L chain mutation status as well as mRNA expression of selected genes (e.g., NOS1 and NOS2). These studies are not complete, but we uncovered important information regarding the biology of the CLL cells (Reference 90). Recent studies have demonstrated intraclonal mutational diversification and ongoing class switching in the heavy chains of CLL cells and have introduced the possibility that individual CLL cells can continue to differentiate. To investigate intraclonal mutational diversification of individual CLL cells, we examined the heavy and light chains from the DNA of singly sorted cells (sorted for CD19+, CD5+, and CD27+ phenotype (Reference 90). Single cells were subjected to 50 cycles of whole genome amplification with random 15-mer primers. Aliquots of these PCR products were used in nested PCR to amplify rearranged Ig genes. H and L chains from 19 single CLL cells from the same patient were amplified and sequenced. This patient had been diagnosed 3 years earlier with CLL Rai stage 0 and had never been treated. All 19H chains sequenced were most similar to VH4-59 and kappa L chains most similar to Vκ1 family member L12. The 19 H chains shared 14 common mutations and K chains shared 17 common mutations from the germline sequences. There were 7 other H chain mutations and 5 other κ chain mutations that defined 7 subgroups of the CLL clone. Genealogical analysis of these subgroups showed that the mutational status of each subgroup was associated with additional sequential mutations. These subgroups shared 31 mutation in common and formed a genealogical tree with 20 unshared mutations. This strongly supports a model of intraclonal mutational diversification in the H and L chains, and supports the idea that continuing somatic mutation plays a role in the evolution of the CLL clone (Reference 90).

In summary, while our work shows that exogenous NO in high amounts may be pro-apoptotic for CLL cells (Reference 40), NO produced endogenously inhibits CLL cell apoptosis and cell death (References 9 and 11) (Reference 106). Furthermore, we found that CLL cells expressed NOS1, and that NOS inhibitors induced CLL cell apoptosis. The ability of NOS inhibitors to induce CLL cell apoptosis was directly related to the avidity of NOS inhibitor binding to NOS1 and was related to the hydrophobicity of these compounds (Reference 106).

While this invention has been described as having preferred sequences, ranges, steps, materials, components, or designs, it is understood that it includes further modifications, variations, uses and/or adaptations thereof following in general the principle of the invention, and including such departures from the present disclosure as those come within the known or customary practice in the art to which the invention pertains, and as may be applied to the central features hereinbeforesetforth, and fall within the scope of the invention and of the limits of the appended claims.

### REFERENCES

The following references, and those cited or discussed herein, are hereby incorporated herein in their entirety by reference.
1. Magrinat G, Mason S N, Shami P J and Weinberg J B. Nitric oxide modulation of human leukemia cell differentiation and gene expression. Blood 80:1880-4, 1992.
2. Kipps T J. Chronic lymphocytic leukemia. Current Opin Hematol 5:244-53, 1998.
3. Johnston J B. Chronic lymphocytic leukemia. In: Lee G R, Foerster J, Lukens J, Paraskevas F, Greer J P and Rodgers G M, eds. Wintrobe's Clinical Hematology (ed Tenth). Baltimore: Williams & Wilkins; 1999:2405-27.
4. Keating M J, Flinn I, Jain V, Binet J L, Hillmen P, Byrd J, Albitar M, Brettman L, Santabarbara P, Wacker B and Rai K R. Therapeutic role of alemtuzumab (Campath-1H) in patients who have failed fludarabine: results of a large international study. Blood 99:3554-61, 2002.
5. Mannick J B, Miao X Q and Stamler J S. Nitric Oxide Inhibits Fas-Induced Apoptosis. J Biol Chem 272:24125-8, 1997.
6. Genaro A M, Hortelano S, Alvarez A, Martinez C and Bosca L. Splenic B lymphocyte programmed cell death is prevented by nitric oxide release through mechanisms involving sustained Bcl-2 levels. J Clin Invest 95:1884-90, 1995.
7. Mannick J B, Asano K, Izumi K, Kieff E and Stamler J S. Nitric oxide produced by human B lymphocytes inhibits apoptosis and Epstein-Barr virus reactivation. Cell 79:1137-46, 1994.
8. Nicotera P, Brune B and Bagetta G. Nitric oxide: inducer or suppressor of apoptosis? Trends Pharmacol Sci 18:189-90, 1997.
9. Levesque M C, Adams D J, Misukonis M A, Flowers J, Silber R and Weinberg J B. Detection of inducible nitric oxide synthase (NOS2) mRNA, antigen and enzyme activity in leukemia cells from patients with CLL. Blood 92 (Suppl 1):431a (abstract), 1998.
10. Levesque M C, Misukonis M A, O'Loughlin C W, Chen Y, Beasley B E, Wilson D L, Adams D J, Silber R and Weinberg J B. IL-4 and interferon gamma regulate expression of inducible nitric oxide synthase in chronic lymphocytic leukemia cells. Leukemia 17:442-50, 2003.
11. Zhao H X, Dugas N, Mathiot C, Delmer A, Dugas B, Sigaux F and Kolb J P. B-cell chronic lymphocytic leukemia cells express a functional inducible nitric oxide synthase displaying anti-apoptotic activity. Blood 92:1031-43, 1998.
12. Borrello M A and Phipps R P. The B/macrophage cell: an elusive link between CD5+ B lymphocytes and macrophages [see comments]. Immunol Today 17:471-5, 1996.
13. Dohner H, Stilgenbauer S, Benner A, Leupolt E, Krober A, Bullinger L, Dohner K, Bentz M and Lichter P. Genomic aberrations and survival in chronic lymphocytic leukemia. N Eng J Med 343:1910-6, 2000.
14. Hamblin T J, Davis Z, Gardiner A, Oscier D G and Stevenson F K. Unmutated Ig V-H genes are associated with a more aggressive form of chronic lymphocytic leukemia. Blood 94:1848-54, 1999.
15. Damle R N, Wasil T, Fais F, Ghiotto F, Valetto A, Allen S L, Buchbinder A, Budman D, Dittmar K, Kolitz J, Lichtman S M, Schulman P, Vinciguerra V P, Rai K R, Ferrarini M and Chiorazzi N. Ig V gene mutation status and CD38 expression as novel prognostic indicators in chronic lymphocytic leukemia. Blood 94:1840-7, 1999.
16. Klein U, Tu Y H, Stolovitzky G A, Mattioli M, Cattoretti G, Husson H, Freedman A, lnghirami G, Cro L, Baldini L, Neri A N, Califano A and Dalla-Favera R. Gene expression profiling of B cell chronic lymphocytic leukemia reveals a homogeneous phenotype related to memory B cells. J Exp Med 194:1625-38, 2001.
17. Rosenwald A, Alizadeh A A, Widhopf G, Simon R, Davis R E, Yu X, Yang L M, Pickeral O K, Rassenti L Z, Powell J, Botstein D, Byrd J C, Grever M R, Cheson B D, Chiorazzi N, Wilson W H, Kipps T J, Brown P O and Staudt L M. Relation of gene expression phenotype to immunoglobulin mutation genotype in B cell chronic lymphocytic leukemia. J Exp Med 194:1639-47, 2001.
18. Jelinek D F, Tschumper R C, Stolovitzky G A, Iturria S J, Tu Y, Lepre J, Shah N and Kay N E. Identification of a global gene expression signature of B-chronic lymphocytic leukemia. Mol Cancer Res 1:346-61, 2003.
19. Crespo M, Bosch F, Villamor N, Bellosillo B, Colomer D, Rozman M, Marce S, Lopez-Guillermo A, Campo E and Montserrat E. ZAP-70 expression as a surrogate for immunoglobulin-variable-region mutations in chronic lymphocytic leukemia. N Engl J Med 348:1764-75, 2003.
20. Wiestner A, Rosenwald A, Barry T S, Wright G, Davis R E, Henrickson S E, Zhao H, Ibbotson R E, Orchard J A, Davis Z, Stetler-Stevenson M, Raffeld M, Arthur D C, Marti G E, Wilson W H, Hamblin T J, Oscier D G and Staudt L M. ZAP-70 expression identifies a chronic lymphocytic leukemia subtype with unmutated immunoglobulin genes, inferior clinical outcome, and distinct gene expression profile. Blood 101:4944-51, 2003.
21. Shanafelt T D, Geyer S M and Kay N E. Prognosis at diagnosis: integrating molecular biologic insights into clinical practice for patients with CLL. Blood 103:1202-10, 2004.
22. Caligaris-Cappio F and Hamblin T J. B-cell chronic lymphocytic leukemia: a bird of a different feather. J Clin Oncol 17:399-408, 1999.
23. Jurlander J. The cellular biology of B-cell chronic lymphocytic leukemia. Crit. Rev Oncol-Hematol 27:29-52, 1998.
24. Frank D A, Mahajan S and Ritz J. B lymphocytes from patients with chronic lymphocytic leukemia contain signal transducer and activator of transcription (STAT) 1 and STAT3 constitutively phosphorylated on serine residues. J Clin Invest 100:3140-8, 1997.
25. Frank D A, Mahajan S and Ritz J. Fludarabine-induced immunosuppression is associated with inhibition of STAT1 signaling. Nature Med 5:444-7, 1999.
26. Mohammad R M, Mohamed A N, Hamdan M Y, Vo T, Chen B, Katato K, Abubakr Y A, Dugan M C and al-Katib A. Establishment of a human B-CLL xenograft model: utility as a preclinical therapeutic model. Leukemia 10:130-7, 1996.
27. Bichi R, Shinton S A, Martin E S, Koval A, Calin G A, Cesari R, Russo G, Hardy R R and Croce C M. Human chronic lymphocytic leukemia modeled in mouse by targeted TCL1 expression. Proc Natl Acad Sci USA 99:6955-60, 2002.
28. Virgilio L, Narducci M G, Isobe M, Billips L G, Cooper M D, Croce C M and Russo G. Identification of the TCL1 gene involved in T-cell malignancies. Proc Natl Acad Sci USA 91:12530-4, 1994.
29. Virgilio L, Lazzeri C, Bichi R, Nibu K, Narducci M G, Russo G, Rothstein J L and Croce C M. Deregulated expression of TCL1 causes T cell leukemia in mice. Proc Natl Acad Sci USA 95:3885-9, 1998.
30. Stamler J S, Singel D J and Loscalzo J. Biochemistry of nitric oxide and its redox-activated forms. [Review]. Science 258:1898-902, 1992.
31. Granger D L, Anstey N M, Miller W C and Weinberg J B. Measuring nitric oxide production in human clinical studies [Review]. Meth Enz 301:49-61, 1999.
32. Fricker S P. Nitrogen monoxide-related disease and nitrogen monoxide scavengers as potential drugs. In: Sigel A and Sigel H, eds. Metal ions in biological systems. Basel: Marcel Dekker, Inc.; 1999:665-721.
33. Weinberg J B. Nitric oxide production and nitric oxide synthase type 2 expression by human mononuclear phagocytes: a review. Molecular Med 4:577-91, 1998.
34. Reiling N, Kroncke R, Ulmer A J, Gerdes J, Flad H D and Hauschildt S.
Nitric oxide synthase: expression of the endothelial, Ca2+/calmodulin-dependent isoform in human B and T lymphocytes. Eur J Immunol 26:511-6, 1996.
35. Mannick J B, Hausladen A, Liu L M, Hess D T, Zeng M, Miao Q X, Kane L S, Gow A J and Stamler J S. Fas-induced caspase denitrosylation. Science 284:6514, 1999.
36. Mendes R V, Martins A R, de Nucci G, Murad F and Soares F A. Expression of nitric oxide synthase isoforms and nitrotyrosine immunoreactivity by B-cell non-Hodgkin's lymphomas and multiple myeloma. Histopathology 39:172-8, 2001.
37. Hibbs J B, Jr, Taintor R R, Vavrin Z and Rachlin E M. Nitric oxide: a cytotoxic activated macrophage effector molecule [published erratum appears in Biochem Biophys Res Commun 1989 Jan. 31; 158(2):624]. Biochem Biophys Res Commun 157:87-94, 1988.
38. Fehsel K, Kroncke K D, Meyer K L, Huber H, Wahn V and Kolbbachofen V. Nitric Oxide Induces Apoptosis In Mouse Thymocytes. J Immunol 155:2858-65, 1995.
39. Shami P J, Sauls D L and Weinberg J B. Schedule and concentration-dependent induction of apoptosis in leukemia cells by nitric oxide. Leukemia 12:1461-6, 1998.
40. Adams D J, Levesque M C, Weinberg J B, Smith K L, Flowers J L, Moore J, Colvin O M and Silber R. Nitric oxide enhancement of fludarabine cytotoxicity for B-CLL lymphocytes. Leukemia 15:1852-9, 2001.
41. Shami P J, Moore J O, Gockerman J P, Hathorn J W, Misukonis M A and Weinberg J B. Nitric oxide modulation of the growth and differentiation of freshly isolated acute non-lymphocytic leukemia cells. Leuk Res 19:527-33, 1995.
42. Mohr S, McCormick T S and Lapetina E G. Macrophages Resistant to Endogenously Generated Nitric Oxide-Mediated Apoptosis Are Hypersensitive to Exogenously Added Nitric Oxide Donors—Dichotomous Apoptotic Response Independent Of Caspase 3 and Reversal By the Mitogen-Activated Protein Kinase Kinase (Mek) Inhibitor Pd 098059. Proc Natl Acad Sci USA 95:5045-50, 1998.
43. Reed J C. Caspases and cytokines: roles in inflammation and autoimmunity. Adv Immunol 265-299, 1999.
44. Li J R, Billiar T R, Talanian R V and Kim Y M. Nitric Oxide Reversibly Inhibits Seven Members Of the Caspase Family Via S-Nitrosylation. Biochem Biophys Res Commun 240:419-24, 1997.
45. Kitada S, Andersen J, Akar S, Zapata J M, Takayama S, Krajewski S, Wang H G, Zhang X, Bullrich F, Croce C M, Rai K, Hines J and Reed J C. Expression of apoptosis-regulating proteins in chronic lymphocytic leukemia: correlations with In vitro and In vivo chemoresponses. Blood 91:3379-89, 1998.
46. King D, Pringle J H, Hutchinson M and Cohen G M. Processing/activation of caspases, -3 and -7 and -8 but not caspase-2, in the induction of apoptosis in B-chronic lymphocytic leukemia cells. Leukemia 12:1553-60, 1998.
47. Forrester K, Ambs S, Lupold S E, Kapust R B, Spillare E A, Weinberg W C, Felley-Bosco E, Wang X W, Geller D A, Tzeng E, Billiar T R and Harris C C. Nitric oxide-induced p53 accumulation and regulation of inducible nitric oxide synthase expression by wild-type p53. Proc Natl Acad Sci USA 93:2442-7, 1996.
48. Ambs S, Ogunfusika M O, Merriam W G, Bennett W P, Billiar T R and Harris C C. Up-Regulation of Inducible Nitric Oxide Synthase Expression in Cancer-Prone P53 Knockout Mice. Proc Natl Acad Sci USA 95:8823-8, 1998.
49. Ambs S, Bennett W P, Merriam W G, Ogunfusika M O, Oser S M, Harrington A M, Shields P G, Felley-Bosco E, Hussain S P and Harris C C. Relationship between p53 mutations and inducible nitric oxide synthase expression in human colorectal cancer. J Natl Cancer Inst 91:86-8, 1999.
50. Andoh T, Lee S Y and Chiueh C C. Preconditioning regulation of bcl-2 and p66shc by human NOS1 enhances tolerance to oxidative stress. Faseb J 14:2144-6, 2000.
51. Thippeswamy T, McKay J S and Morris R. Bax and caspases are inhibited by endogenous nitric oxide in dorsal root ganglion neurons in vitro. Eur J Neurosci 14:1229-36, 2001.
52. Nathan C and Xie Q-W. Regulation of biosynthesis of nitric oxide. J Biol Chem 269:13725-8, 1994.
53. Levesque M C, Hobbs M R, O'Loughlin C W, Chancellor J A, Tkachuk A N, Booth J, Patch K B, Pole A R, Fernandez C A, Burch L H, Mwaikambo E D, Granger D L, Anstey N M, Kaplan N L and Weinberg J B. Identification of single nucleotide polymorphisms (SNPs) and haplotypes, and analysis of a recombination hot spot in the human nitric oxide synthase type 2 (NOS2) promoter. Submitted, 2004.
54. Hobbs M R, Udhayakumar V, Levesque M C, Booth J, Roberts J M, Tkachuk A N, Pole A, Coon H, Kariuki S, Nahlen B L, Mwaikambo E D, Lal A L, Granger D L, Anstey N M and Weinberg J B. A new NOS2 promoter polymorphism associated with increased nitric oxide production and protection from severe malaria in Tanzanian and Kenyan children. Lancet 360:1468-75, 2002.
55. Rodriguez-Pascual F, Hausding M, Ihrig-Biedert I, Furneaux H, Levy A P, Forstermann U and Kleinert H. Complex contribution of the 3′-untranslated region to the expressional regulation of the human inducible nitric-oxide synthase gene—Involvement of the RNA-binding protein HuR. J Biol Chem 275:26040-9, 2000.
56. Christopherson K S and Bredt D S. Nitric Oxide In Excitable Tissues—Physiological Roles and Disease. J Clin Invest 100:2424-9, 1997.
57. Eissa N T, Yuan J W, Haggerty C M, Choo E K, Palmer C D and Moss J. Cloning and Characterization of Human Inducible Nitric Oxide Synthase Splice Variants—a Domain, Encoded By Exons 8 and 9, Is Critical For Dimerization. Proc Natl Acad Sci USA 95:7625-30, 1998.
58. Gross S S and Levi R. Tetrahydrobiopterin synthesis. An absolute requirement for cytokine-induced nitric oxide generation by vascular smooth muscle. J Biol Chem 267:25722-9, 1992.
59. Wang Y, Newton D C and Marsden P A. Neuronal NOS: gene structure, mRNA diversity, and functional relevance. Crit. Rev Neurobiol 13:2143, 1999.
60. Hall A V, Antoniou H, Wang Y, Cheung A H, Arbus A M, Olson S L, Lu W C, Kau C L and Marsden P A. Structural organization of the human neuronal nitric oxide synthase gene (NOS1). J Biol Chem 269:33082-90, 1994.
61. Wang Y, Newton D C, Miller T L, Teichert A M, Phillips M J, Davidoff M S and Marsden P A. An alternative promoter of the human neuronal nitric oxide synthase gene is expressed specifically in Leydig cells. Am J Pathol 160:369-80, 2002.
62. Saur D, Vanderwinden J M, Seidler B, Schmid R M, De Laet M H and Allescher H D. Single-nucleotide promoter polymorphism alters transcription of neuronal nitric oxide synthase exon 1c in infantile hypertrophic pyloric stenosis. Proc Natl Acad Sci USA 2, 2004.
63. Wang Y, Newton D C, Robb G B, Kau C L, Miller T L, Cheung A H, Hall A V, VanDamme S, Wilcox J N and Marsden P A. RNA diversity has profound effects on the translation of neuronal nitric oxide synthase. Proc Natl Acad Sci USA 96:12150-5, 1999.
64. Newton D C, Bevan S C, Choi S, Robb G B, Millar A, Wang Y and Marsden P A. Translational regulation of human neuronal nitric-oxide synthase by an alternatively spliced 5′-untranslated region leader exon. J Biol Chem 278:636-44, 2003.
65. Brenman J E, Xia H, Chao D S, Black S M and Bredt D S. Regulation of neuronal nitric oxide synthase through alternative transcripts. Dev Neurosci 19:224-31, 1997.
66. Forstermann U, Boissel J P and Kleinert H. Expressional control of the ‘constitutive’ isoforms of nitric oxide synthase (NOS I and NOS III). Faseb J 12:773-90, 1998.
67. Chesler D A, McCutcheon J A and Reiss C S. Posttranscriptional Regulation of Neuronal Nitric Oxide Synthase Expression by IFN-gamma. J Interferon Cytokine Res 24:141-9, 2004.
68. Schmidt H H, Hofmann H and Ogilvie P. Regulation and dysregulation of constitutive nitric oxide synthases types I and III. Curr Top Microbiol Immunol 196:75-86, 1995.
69. Hibbs J B, Jr, Vavrin Z and Taintor R R. L-arginine is required for expression of the activated macrophage effector mechanism causing selective metabolic inhibition in target cells. J Immunol 138:550-65, 1987.
70. Babu B R and Griffith O W. Design of isoform-selective inhibitors of nitric oxide synthase [Review]. Current Opinion in Chemical Biology 2:491-500, 1998.
71. Chabrier P E, Demerle-Pallardy C and Auguet M. Nitric oxide synthases: targets for therapeutic strategies in neurological diseases [Review]. Cellular & Molecular Life Sciences 55:1029-35, 1999.
72. Blasko E, Glaser C B, Devlin J J, Xia W, Feldman R I, Polokoff M A, Phillips G B, Whitlow M, Auld D S, McMillan K, Ghosh S, Stuehr D J and Parkinson J F. Mechanistic studies with potent and selective inducible nitric-oxide. J Biol Chem 277:295-302, 2002.
73. Bakker J, Grover R, McLuckie A, Holzapfel L, Andersson J, Lodato R, Watson D, Grossman S, Donaldson J and Takala J. Administration of the nitric oxide synthase inhibitor NG-methyl-L-arginine hydrochloride (546C88) by intravenous infusion for up to 72 hours can promote the resolution of shock in patients with severe sepsis: results of a randomized, double-blind, placebo-controlled multicenter study (study no. 144-002). Crit Care Med 32:1-12, 2004.
74. Tepper S J, Rapoport A and Sheftell F. The pathophysiology of migraine. Neurolog 7:279-86, 2001.
75. Hansel T T, Kharitonov S A, Donnelly L E, Erin E M, Currie M G, Moore W M, Manning P T, Recker D P and Barnes P J. A selective inhibitor of inducible nitric oxide synthase inhibits exhaled breath nitric oxide in healthy volunteers and asthmatics. FASEB J 17:1298-300, 2003.
76. Sonoki T, Matsuzaki H, Nagasaki A, Hata H, Yoshida M, Matsuoka M, Kuribayashi N, Kimura T, Harada N, Takatsuki K, Mitsuya H and Mori M. Detection of inducible nitric oxide synthase (iNOS) mRNA by RT-PCR in ATL patients and HTLV-1 infected cell lines: clinical features and apoptosis by NOS inhibitor. Leukemia 13:713-8, 1999.
77. Kitagawa M, Takahashi M, Yamaguchi S, Inoue M, Ogawa S, Hirokawa K and Kamiyama R. Expression of inducible nitric oxide synthase (NOS) in bone marrow cells of myelodysplastic syndromes. Leukemia 13:699-703, 1999.
78. Roman V, Zhao H, Fourneau J M, Marconi A, Dugas N, Dugas B, Sigaux F and Kolb J P. Expression of a functional inducible nitric oxide synthase in hairy cell leukaemia and ESKOL cell line. Leukemia 14:696-705, 2000.
79. Marwick C. Link found between Agent Orange and chronic lymphocytic leukaemia. British Medical Journal 326:242, 2003.
80. Frumkin K. Agent orange and cancer: an overview for clinicians. CA Cancer J Clin 53:245-55, 2003.
81. Salvucci O, Carsana M, Bersani I, Tragni G and Anichini A. Antiapoptotic role of endogenous nitric oxide in human melanoma cells. Cancer Res 61:318-26, 2001.
82. Thomsen L L, Miles D W, Happerfield L, Bobrow L G, Knowles R G and Moncada S. Nitric oxide synthase activity in human breast cancer. Br J Cancer 72:41-4, 1995.
83. Weinberg J B and Hibbs J B, Jr. Endocytosis of red blood cells or haemoglobin by activated macrophages inhibits their tumoricidal effect. Nature 269:245-7, 1977.
84. Weinberg J B. Tumor cell killing by phorbol ester-differentiated human leukemia cells. Science 213:655-7, 1981.
85. Shami P J and Weinberg J B. Differential effects of nitric oxide on erythroid and myeloid colony growth from CD34(+) human bone marrow cells. Blood 87:977-82, 1996.
86. Levesque M C, O'Loughlin C W and Weinberg J B. Use of serum-free media to minimize apoptosis of chronic lymphocytic leukemia cells during in vitro culture. Leukemia 15:1305-7, 2001.
87. Levesque M C, Chen Y, Beasley B E, O'Loughlin C W, Misukonis M A, Gockerman J P, Moore J O and Weinberg J B. Chronic lymphocytic leukemia cell CD38 expression and inducible nitric oxide synthase expression are associated with serum IL-4 levels. Submitted, 2003.
88. Levesque M C, Chen Y, Beasley B E, O'Loughlin C W, Gockerman J P, Moore J O and Ghosh D K. Chronic lymphocytic leukemia cells (CLL) express the neuronal isoform of nitric oxide synthase (NOS1), and NOS1-specific inhibitors induce CLL cell death. Blood 102:353b (abstract), 2003.
89. Vaccaro G M, Moore J O, Gockerman J P and Weinberg J B. Protein phosphatase inhibitors as potent cytotoxic agents for B-cell CLL in vitro. Blood 102:306b (abstract), 2003.
90. Volkheimer A D, Weinberg J B, Beasley B E and Levesque M C. A single cell cell analysis of immunoglobulin genes in chronic lymphocytic leukemia (CLL): progressive somatic mutations in the immunoglobulin heavy and light chains contribute to intraclonal diversification in CLL. Blood 102:666a (abstract), 2003.
91. Weinberg J B. Nitric oxide as an inflammatory mediator in autoimmune MRL-Ipr/Ipr mice. Envir Health Persp 106:1131-7, 1998.
92. Weinberg J B. Nitric oxide synthase 2 and cyclooxygenase 2 interactions in inflammation. Immunol Res 22:319-41, 2000.
93. Anstey N M, Weinberg J B and Granger DL. Nitric oxide and nitric oxide synthase type 2 in severe malaria. In: Fang F ed. Nitric oxide and infection. New York: Kluwer Academic/Plenum Publishing:311-41, 1999.
94. Weinberg J B. Mononuclear phagocytes. In: Lee G R, ed. Wintrobe's clinical hematology (ed 11th). Philadelphia: Williams and Wilkins; 2003.
95. Weinberg J B, Granger D L, Pisetsky D S, Seldin M F, Misukonis M A, Mason S N, Pippen A M, Ruiz P, Wood E R and Gilkeson G S. The role of nitric oxide in the pathogenesis of spontaneous murine autoimmune disease: increased nitric oxide production and nitric oxide synthase expression in MRL-Ipr/Ipr mice, and reduction of spontaneous glomerulonephritis and arthritis by orally administered NG-monomethyl-L-arginine. J Exp Med 179:651-60, 1994.
96. Gilkeson G S, Mudgett J S, Seldin M F, Ruiz P, Alexander A A, Misukonis M A, Pisetsky D S and Weinberg J B. Clinical and serologic manifestations of autoimmune disease In MRL-Ipr/Ipr mice lacking nitric oxide synthase type 2. J Exp Med 186:365-73, 1997.
97. Keng T, Privalle C T, Gilkeson G S and Weinberg J B. Peroxynitrite and decreased catalase in autoimmune mice. Molecular Med In press, 2000.
98. Perkins D J, St Clair E W, Misukonis M A and Weinberg J B. Reduction of NOS2 overexpression in rheumatoid arthritis patients treated with anti-tumor necrosis factor alpha monoclonal antibody (cA2). Arth Rheum 41:2205-10, 1998.
99. Muscato J J, Haney A F and Weinberg J B. Sperm phagocytosis by human peritoneal macrophages: a possible cause of infertility in endometriosis. Am J Obstet Gynecol 144:503-10, 1982.
100. Osborn B H, Haney A F, Misukonis M A and Weinberg J B. Inducible nitric oxide synthase expression by peritoneal macrophages in endometriosis-associated infertility. Fertil Steril 77:46-51, 2002.
101. Sharara A I, Perkins D J, Misukonis M A, Chan S U, Dominitz J A and Weinberg J B. Interferon (IFN)-alpha activation of human blood mononuclear cells in vitro and in vivo for nitric oxide synthase (NOS) type 2 mRNA and protein expression—possible relationship of induced NOS2 to the anti-hepatitis C effects of IFN-alpha in vivo. J Exp Med 186:1495-502, 1997.
102. Anstey N M, Weinberg J B, Hassanali M, Mwaikambo E D, Manyenga D, Misukonis M A, Arnelle D R, Hollis D, McDonald M I and Granger D L. Nitric oxide in Tanzanian children with malaria. Inverse relationship between malaria severity and nitric oxide production/nitric oxide synthase type 2 expression. J Exp Med 184:557-67, 1996.
103. Lopansri B K, Anstey N M, Weinberg J B, Stoddard G J, Hobbs M R, Levesque M C, Mwaikambo E D and Granger D L. Low plasma arginine concentrations in children with cerebral malaria and decreased nitric oxide production. Lancet 361:676-8, 2003.
104. Cheson B D, Bennett J M, Grever M, Kay N, Keating M J, O'Brien S and Rai K R. National Cancer Institute-sponsored Sorking Group guidelines for chronic lymphocytic leukemia: revised guidelines for diagnosis and treatment. Blood 87:4990-7, 1996.
105. Brouwer, M, W. Chamulitrat, G. Ferruzzi, D. L. Sauls, and J. B. Weinberg, Nitric oxide interactions with cobalamins. Biochemical and functional consequences. Blood 88: 1857-1864, 1996.)
106. Levesque M C, Ghosh D K, Beasley B E, Chen Y, Volkheimer A D, O'Loughlin C W, Gockerman J P, Moore J O, Weinberg J B. CLL cell apoptosis induced by nitric oxide synthase inhibitors: correlation with lipid solubility and NOS1 dissociation constant. Leuk Res 32: 1061-1070, 2008.

What is claimed is:

1. A method of inducing apoptosis or cell death in a cancer cell, comprising:
inhibiting production of nitric oxide in a cancer cell.

2. The method of claim 1, wherein:
the production of nitric oxide is inhibited by interfering with the activity or expression of a nitric oxide synthase.

3. The method of claim 2, wherein:
the activity or expression of the nitric oxide synthase is regulated by a NOS inhibitor or a NOS expression inhibitor.

4. The method of claim 3, wherein:
the NOS inhibitor comprises an isoform-specific inhibitor.

5. The method of claim 4, wherein:
the NOS inhibitor comprises a NOS1-specific inhibitor.

6. The method of claim 5, wherein:
the NOS inhibitor comprises at least one member selected from the group consisting of N-[4-(2-{[(3-chlorophenyl)methyl]amino}ethyl)phenyl]-2-thiophenecarboximide dihydrochloride, 7-nitroindazole, 1-(2-trifluoromethylphenyl)imidazole, [N⁵-(1-imino-3-butenyl)-L-ornithine], 3-bromo-7-nitroindazole, and S-ethyl-N-[4-(trifluoromethyl)phenyl)isothiourea HCl.

7. The method of claim 3, wherein:
the NOS expression inhibitor comprises a glucocorticoid.

8. The method of claim 2, wherein:
the nitric oxide synthase comprises NOS1.

9. The method of claim 2, wherein:
the nitric oxide synthase is selected from the group consisting of NOS1, NOS2 and NOS3.

10. The method of claim 1, wherein:
the cancer cell comprises a lymphocytic leukemia cell.

11. A method of inducing apoptosis or cell death in a leukemia cell, comprising:
subjecting a leukemia cell to a NOS1-specific inhibitor.

12. The method of claim 11, wherein:
the NOS1-specific inhibitor comprises at least one member selected from the group consisting of N-[4-(2-{[(3-chlorophenyl)methyl]amino}ethyl)phenyl]-2-thiophenecarboximide dihydrochloride, 7-nitroindazole, 1-(2-trifluoromethylphenyl)imidazole, [N⁵-(1-imino-3-butenyl)-L-ornithine], 3-bromo-7-nitroindazole, and S-ethyl-N-[4-(trifluoromethyl)phenyl)isothiourea HCl.

13. The method of claim 12, wherein:
the leukemia cell comprises a lymphocytic leukemia cell.

14. A method of treating leukemia, comprising:
administering to a subject in need thereof an effective amount of a NOS1-specific inhibitor for inhibiting the activity or expression of a nitric oxide synthase in an affected cell.

15. The method of claim 14, wherein:
the NOS1-specific inhibitor comprises at least one member selected from the group consisting of N-[4-(2-{[(3-chlorophenyl)methyl]amino}ethyl)phenyl]-2-thiophenecarboximide dihydrochloride, 7-nitroindazole, 1-(2-trifluoromethylphenyl)imidazole, [N⁵-(1-imino-3-butenyl)-L-ornithine], 3-bromo-7-nitroindazole, and S-ethyl-N-[4-(trifluoromethyl)phenyl)isothiourea HCl.

16. The method of claim 15, wherein:
the NOS1-specific inhibitor is administered intravenously, orally, or subcutaneously.

17. The method of claim 16, wherein:
the leukemia comprises chronic lymphocytic leukemia.

18. The method of claim 14, wherein:
the NOS1-specific inhibitor comprises a glucocorticoid.
